SEQUENCE LISTING (14 sequences)

SEQ ID NO: 1 - DNA, 21 bp, synthetic construct
  misc_feature 1..20: note = CMV promoter forward primer
  source 1..21: mol_type = other DNA
tcacggggatttccaagtctc

SEQ ID NO: 2 - DNA, 22 bp, synthetic construct
  misc_feature 1..22: note = CMV promoter reverse primer
  source 1..22: mol_type = other DNA
aatggggcggagttgttacgac

SEQ ID NO: 3 - DNA, 22 bp, synthetic construct
  misc_feature 1..22: note = CMV promoter probe
  source 1..22: mol_type = other DNA
  misc_feature 1: note = 56-FAM
  misc_feature 10: note = ZEN
  misc_feature 22: note = 3IaBkFQ
aaacaaactcccattgacgtca

SEQ ID NO: 4 - DNA, 20 bp, synthetic construct
  misc_feature 1..20: note = Nucleoplasmids forward primer
  source 1..20: mol_type = other DNA
atgtctgataatggacccca

SEQ ID NO: 5 - DNA, 18 bp, synthetic construct
  misc_feature 1..18: note = Nucleoplasmids reverse primer
  source 1..18: mol_type = other DNA
ttaggcctgagttgagtc

SEQ ID NO: 6 - DNA, 8910 bp, synthetic construct
  misc_feature 1..8910: note = Vector Hgpm2
  source 1..8910: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattctagactgccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggagacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagaacctgcagggccagatggtgcaccaggccatctccccccgcaccctgaacgcctgggtgaaggtggtggaggagaaggccttctcccccgaagtcatccccatgttctccgccctgtccgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggctccgacatcgccggcaccacctccaccctgcaagagcagatcggctggatgacccacaacccccccatccccgtgggcgagatctacaagcgctggatcatcctgggcctgaacaagatcgtgcgcatgtactcccccacctccatcctggacatccgccagggccccaaggagcccttccgcgactacgtggaccgcttctacaagaccctgcgcgccgagcaggcctcccaggaggtaaagaactggatgaccgagaccctgctggtgcagaacgccaaccccgactgcaagaccatcctgaaggccctgggccccggcgccaccctggaggagatgatgaccgcctgccagggcgtgggcggccccggccacaaggcccgcgtgctggccgaggccatgtcccaagtcaccaaccccgccaccatcatgatccagaagggcaacttccgcaaccagcgcaagaccgtgaagtgcttcaactgcggcaaggagggccacatcgccaagaactgccgcgccccccgcaagaagggctgctggaagtgcggcaaggagggccaccagatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggtggccagctgaaggaggccctgctggacaccggcgccgacgacaccgtgctggaggagatgaacctgcccggccgctggaagcccaagatgatcggcggcatcggcggcttcatcaaagtccgccagtacgaccagatcctgatcgagatctgcggccacaaggccatcggcaccgtgctggtgggccccacccccgtgaacatcatcggccgcaacctgctgacccagatcggctgcaccctgaacttccccatctcccccatcgagaccgtgcccgtgaagctgaagcccggcatggacggccccaaagtcaagcagtggcccctgaccgaggagaagatcaaggccctggtggagatctgcaccgagatggagaaggagggcaagatctccaagatcggccccgagaacccctacaacacccccgtgttcgccatcaagaagaaggactccaccaagtggcgcaagctggtggacttccgcgagctgaacaagcgcacccaggacttctgggaggtgcagctgggcatcccccaccccgccggcctgaagcagaagaagtccgtgaccgtgctggacgtgggcgacgcctacttctccgtgcccctggacaaggacttccgcaagtacaccgccttcaccatcccctccatcaacaacgagacccccggcatccgctaccagtacaacgtgctgccccagggctggaagggctcccccgccatcttccagtgctccatgaccaagatcctggagcccttccgcaagcagaaccccgacatcgtgatctaccagtacatggacgacctgtacgtgggctccgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggactcctggaccgtgaacgacatccagaagctggtgggcaagctgaactgggcctcccagatctacgccggcatcaaagtccgccagctgtgcaagctgctgcgcggcaccaaggccctgaccgaggtggtgcccctgaccgaggaggccgagctggagctggccgagaaccgcgagatcctgaaggagcccgtgcacggcgtgtactacgacccctccaaggacctgatcgccgagatccagaagcagggccagggccagtggacctaccagatctaccaggagcccttcaagaacctgaagaccggcaaatacgcccgcatgaagggcgcccacaccaacgacgtgaagcagctgaccgaggccgtgcagaagatcgccaccgagtccatcgtgatctggggcaagactcccaagttcaagctgcccatccagaaggagacctgggaggcctggtggaccgagtactggcaggccacctggatccccgagtgggagttcgtgaacaccccccccctggtgaagctgtggtaccagctggagaaggagcccatcatcggcgccgagaccttctacgtggacggcgccgccaaccgcgagaccaagctgggcaaggccggctacgtgaccgaccgcggccgccagaaggtggtgcccctgaccgacaccaccaaccagaagaccgagctgcaggccatccacctggccctgcaagactccggcctggaggtgaacatcgtgaccgactcccagtatgcattgggcatcatccaggcccagcccgacaagtccgagtccgagctggtgtcccagatcatcgagcagctgatcaagaaggagaaggtgtacctggcctgggtgcccgcccacaagggcatcggcggcaacgagcaggtggacaagctggtgtccgccggcatccgcaaggtgctgttcctggacggcatcgacaaggcccaggaggagcacgagaagtaccactccaactggcgcgccatggcctccgacttcaacctgccccccgtggtggccaaggagatcgtggcctcctgcgacaagtgccagctgaagggcgaggccatgcacggccaggtggactgctcccccggcatctggcagctggactgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccgtgcacaccgacaacggctccaacttcacctccaccaccgtgaaggccgcctgctggtgggccggcatcaagcaggagttcggcatcccctacaacccccagtcccagggcgtgatcgagtccatgaacaaggagctgaagaagatcatcggccaagtccgcgaccaggccgagcacctgaagaccgccgtgcagatggccgtgttcatccacaacttcaagcgcaagggcggcatcggcggctactccgccggcgagcgcatcgtggacatcatcgccaccgacatccagaccaaggagctgcagaagcagatcaccaagatccagaacttccgcgtgtactaccgcgactcccgcgaccccgtgtggaagggccccgccaagctgctgtggaagggcgagggcgccgtggtgatccaggacaactccgacatcaaggtggtgccccgccgcaaggccaagatcatccgcgactacggcaagcagatggccggcgacgactgcgtggcctcccgccaggacgaggactaacacatggaaaagattagtaaaacaccataggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 7 - DNA, 4829 bp, synthetic construct
  misc_feature 1..4829: note = Vector Tat1b
  source 1..4829: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcgaaatggagccagtagatcctagactagagccctggaagcatccaggaagtcagcctaaaactgcttgtaccacttgctattgtaaaaagtgttgctttcattgccaagtttgtttcacaacaaaagccttaggcatctcctatggcaggaagaagcggagacagcgacgaagacctcctcaaggcagtcagactcatcaagtttctctatcaaagcaacccacctcccaaccccgaggggacccgacaggcccgaaggaataggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 8 - DNA, 5900 bp, synthetic construct
  misc_feature 1..5900: note = Vector Rev1b
  source 1..5900: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggccgctcgagcatgcatctagactgccatggcaggaagaagcggagacagcgacgaagacctcctcaaggcagtcagactcatcaagtttctctatcaaagcaacccacctcccaaccccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaataggatccggctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 9369 bp, synthetic construct
  misc_feature 1..9369: note = Vector Luc2-ZsGreen
  source 1..9369: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 10 - DNA, 8327 bp, synthetic construct
  misc_feature 1..8327: note = Vector Spike G614Delta19
  source 1..8327: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgccatgttcgtgttcctggtactccttcctttggtgtcttcccagtgtgtaaatcttaccactcggacccagcttccacccgcctacaccaacagtttcacacgcggcgtctactatcctgacaaggtgtttaggagttcagtcttgcactcaactcaagacttgttcctccctttctttagcaatgtgacgtggtttcatgccattcatgtctccggcacaaacggaacgaagcgctttgataatcctgtgctcccgttcaacgatggagtgtacttcgcgtccacagagaagagcaatatcattcgaggttggatcttcggaacgacactcgactcaaagacgcagtcccttctcatcgtcaataatgccacgaacgtggtcatcaaagtgtgcgagtttcaattctgtaatgatcccttcctgggcgtctattatcacaagaacaacaaatcctggatggagtccgaatttagagtctactccagcgccaacaactgcactttcgaatacgtatcacagccattcttgatggaccttgaaggaaagcagggtaatttcaagaacttgagggagttcgtattcaagaatatcgacgggtactttaagatttatagcaaacacacacccattaatttggtgcgggatcttcctcagggatttagtgctcttgagcctctcgttgacctccctattggcattaacatcacccgctttcaaaccctgttggccctgcatcggtcctacctgacaccgggcgactcaagttccggatggaccgcaggtgccgccgcatactatgtgggctaccttcagccaagaacatttctgctgaaatataatgagaacgggaccattacagatgcggtggattgtgcactcgaccctctgtctgagacgaaatgcacccttaagagcttcacggtggagaaaggcatttatcagacttctaacttcagagttcaacccaccgagtccattgtgcgattcccaaatattacgaatttgtgcccatttggtgaggtcttcaatgctactcgattcgcctcagtttatgcatggaaccgaaagagaatttccaattgtgtggcggactactcagtattgtataatagtgcaagctttagcacattcaaatgttacggcgtgtctccaacgaagctgaacgatctctgtttcacaaacgtttatgcggattccttcgtgattcgcggcgatgaggtccgacagattgcgcctgggcaaacgggtaagatcgctgattacaactataagttgccggacgatttcacaggatgtgtcatagcttggaatagcaataatttggacagtaaggttggcggaaactacaattatttgtacaggttgtttcgcaagtcaaatttgaaaccatttgagagagatatatctacggagatatatcaagccggctctacaccatgtaatggtgtggagggctttaactgctactttccactccagtcatatggtttccaacctacaaatggagtagggtatcaaccgtacagagttgtggtcttgagtttcgaattgctccacgctccagcaacggtatgcggtcctaagaaatccacaaatcttgtgaagaacaagtgcgtaaatttcaacttcaatgggctgactggaacaggcgtgctgactgagagtaacaagaagttcttgcctttccaacaattcgggcgggacatagctgataccactgacgccgtccgcgaccctcagaccctggagattctggacataactccttgttctttcggtggcgtcagtgttatcactcccgggaccaacacctccaaccaagtcgcggtcctctatcaaggcgtcaactgtacggaagtaccggtagccatccatgcggaccaacttacaccgacttggagggtttactctacaggaagcaatgtctttcaaacacgagccgggtgtctgatcggagcagaacacgttaacaacagctacgaatgtgacataccaataggcgcagggatttgtgcttcatatcagacacagaccaatagcccgagcagagcgagtagcgtagcaagccaaagcatcatcgcgtacacgatgagcctcggagcagagaacagcgtcgcgtatagcaataattcaatagctatcccaacaaatttcactatttcggtcaccactgagattctgccggtctccatgaccaagacatccgtcgattgtactatgtacatatgcggcgacagcacggagtgcagtaacttgctccttcagtacggttccttctgtacgcagcttaaccgggcactgacgggtatcgcggtagaacaggacaagaacacacaggaggtcttcgcgcaggtcaaacaaatctacaagacaccacccataaaggacttcggcgggttcaatttcagccaaatcctgccggacccttccaaacctagtaagaggtcattcattgaggatcttctgtttaacaaagttacgcttgcggacgcgggattcattaagcagtatggtgactgccttggagatattgccgccagggatttgatatgtgcacagaaatttaacggcctcaccgttctgccgcctctgctcaccgatgagatgatagcgcagtacacgagcgcactcctggcaggtacaattacaagcggatggacattcggtgcaggagcagcgttgcagataccctttgctatgcagatggcttatcgatttaacgggattggcgtcacgcagaacgtcctttatgagaatcagaaattgattgcaaatcagttcaatagtgctatcggtaagattcaggacagcttgagcagtaccgcgtctgcactgggaaagttgcaggacgtggtgaatcagaatgcacaagcactgaataccttggttaagcaattgagtagcaatttcggcgccatatcaagtgtactgaatgatatcctgtcacggttggacaaggtagaagccgaagttcagattgaccgcttgatcaccgggcgcctccaaagtctgcagacctacgtcacacaacaattgatcagagcagcagagataagagcatctgctaacctggctgccactaagatgtctgaatgtgtgcttgggcagtcaaagagggtagatttctgcggaaagggctaccaccttatgtctttccctcagagcgctccgcatggtgtggtctttctccatgtgacttatgtgcctgctcaagagaagaactttacgacggcgcccgctatatgccatgatggtaaggcgcactttccaagggagggagtgttcgtgtccaacggcactcactggtttgtcacccaacgaaatttctacgagcctcaaattattaccaccgacaatacctttgttagcggtaactgtgacgtcgtaattgggattgttaataatacagtctacgatcctctgcagccggaactggactcctttaaagaggagctggacaaatatttcaagaaccacacatctcctgacgtagatcttggagacataagcggtataaatgcaagtgttgttaacattcagaaagaaatagataggttgaacgaagttgcgaagaaccttaacgagtcactgatagacctccaagagcttgggaagtacgagcaatatatcaagtggccttggtatatttggctcgggttcatagcaggacttatcgctatagtcatggtgactataatgctgtgctgcatgacaagctgctgcagctgtctcaaaggctgttgctcttgcggctcttgctgctaatgaaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 11 - DNA, 3767 bp, synthetic construct
  misc_feature 1..3767: note = Modified sequence from wild type Spike
  source 1..3767: mol_type = other DNA
atgttcgtgttcctggtactccttcctttggtgtcttcccagtgtgtaaatcttaccactcggacccagcttccacccgcctacaccaacagtttcacacgcggcgtctactatcctgacaaggtgtttaggagttcagtcttgcactcaactcaagacttgttcctccctttctttagcaatgtgacgtggtttcatgccattcatgtctccggcacaaacggaacgaagcgctttgataatcctgtgctcccgttcaacgatggagtgtacttcgcgtccacagagaagagcaatatcattcgaggttggatcttcggaacgacactcgactcaaagacgcagtcccttctcatcgtcaataatgccacgaacgtggtcatcaaagtgtgcgagtttcaattctgtaatgatcccttcctgggcgtctattatcacaagaacaacaaatcctggatggagtccgaatttagagtctactccagcgccaacaactgcactttcgaatacgtatcacagccattcttgatggaccttgaaggaaagcagggtaatttcaagaacttgagggagttcgtattcaagaatatcgacgggtactttaagatttatagcaaacacacacccattaatttggtgcgggatcttcctcagggatttagtgctcttgagcctctcgttgacctccctattggcattaacatcacccgctttcaaaccctgttggccctgcatcggtcctacctgacaccgggcgactcaagttccggatggaccgcaggtgccgccgcatactatgtgggctaccttcagccaagaacatttctgctgaaatataatgagaacgggaccattacagatgcggtggattgtgcactcgaccctctgtctgagacgaaatgcacccttaagagcttcacggtggagaaaggcatttatcagacttctaacttcagagttcaacccaccgagtccattgtgcgattcccaaatattacgaatttgtgcccatttggtgaggtcttcaatgctactcgattcgcctcagtttatgcatggaaccgaaagagaatttccaattgtgtggcggactactcagtattgtataatagtgcaagctttagcacattcaaatgttacggcgtgtctccaacgaagctgaacgatctctgtttcacaaacgtttatgcggattccttcgtgattcgcggcgatgaggtccgacagattgcgcctgggcaaacgggtaagatcgctgattacaactataagttgccggacgatttcacaggatgtgtcatagcttggaatagcaataatttggacagtaaggttggcggaaactacaattatttgtacaggttgtttcgcaagtcaaatttgaaaccatttgagagagatatatctacggagatatatcaagccggctctacaccatgtaatggtgtggagggctttaactgctactttccactccagtcatatggtttccaacctacaaatggagtagggtatcaaccgtacagagttgtggtcttgagtttcgaattgctccacgctccagcaacggtatgcggtcctaagaaatccacaaatcttgtgaagaacaagtgcgtaaatttcaacttcaatgggctgactggaacaggcgtgctgactgagagtaacaagaagttcttgcctttccaacaattcgggcgggacatagctgataccactgacgccgtccgcgaccctcagaccctggagattctggacataactccttgttctttcggtggcgtcagtgttatcactcccgggaccaacacctccaaccaagtcgcggtcctctatcaaggcgtcaactgtacggaagtaccggtagccatccatgcggaccaacttacaccgacttggagggtttactctacaggaagcaatgtctttcaaacacgagccgggtgtctgatcggagcagaacacgttaacaacagctacgaatgtgacataccaataggcgcagggatttgtgcttcatatcagacacagaccaatagcccgagcagagcgagtagcgtagcaagccaaagcatcatcgcgtacacgatgagcctcggagcagagaacagcgtcgcgtatagcaataattcaatagctatcccaacaaatttcactatttcggtcaccactgagattctgccggtctccatgaccaagacatccgtcgattgtactatgtacatatgcggcgacagcacggagtgcagtaacttgctccttcagtacggttccttctgtacgcagcttaaccgggcactgacgggtatcgcggtagaacaggacaagaacacacaggaggtcttcgcgcaggtcaaacaaatctacaagacaccacccataaaggacttcggcgggttcaatttcagccaaatcctgccggacccttccaaacctagtaagaggtcattcattgaggatcttctgtttaacaaagttacgcttgcggacgcgggattcattaagcagtatggtgactgccttggagatattgccgccagggatttgatatgtgcacagaaatttaacggcctcaccgttctgccgcctctgctcaccgatgagatgatagcgcagtacacgagcgcactcctggcaggtacaattacaagcggatggacattcggtgcaggagcagcgttgcagataccctttgctatgcagatggcttatcgatttaacgggattggcgtcacgcagaacgtcctttatgagaatcagaaattgattgcaaatcagttcaatagtgctatcggtaagattcaggacagcttgagcagtaccgcgtctgcactgggaaagttgcaggacgtggtgaatcagaatgcacaagcactgaataccttggttaagcaattgagtagcaatttcggcgccatatcaagtgtactgaatgatatcctgtcacggttggacaaggtagaagccgaagttcagattgaccgcttgatcaccgggcgcctccaaagtctgcagacctacgtcacacaacaattgatcagagcagcagagataagagcatctgctaacctggctgccactaagatgtctgaatgtgtgcttgggcagtcaaagagggtagatttctgcggaaagggctaccaccttatgtctttccctcagagcgctccgcatggtgtggtctttctccatgtgacttatgtgcctgctcaagagaagaactttacgacggcgcccgctatatgccatgatggtaaggcgcactttccaagggagggagtgttcgtgtccaacggcactcactggtttgtcacccaacgaaatttctacgagcctcaaattattaccaccgacaatacctttgttagcggtaactgtgacgtcgtaattgggattgttaataatacagtctacgatcctctgcagccggaactggactcctttaaagaggagctggacaaatatttcaagaaccacacatctcctgacgtagatcttggagacataagcggtataaatgcaagtgttgttaacattcagaaagaaatagataggttgaacgaagttgcgaagaaccttaacgagtcactgatagacctccaagagcttgggaagtacgagcaatatatcaagtggccttggtatatttggctcgggttcatagcaggacttatcgctatagtcatggtgactataatgctgtgctgcatgacaagctgctgcagctgtctcaaaggctgttgctcttgcggctcttgctgctaatg

SEQ ID NO: 12 - DNA, 9501 bp, synthetic construct
  misc_feature 1..9501: note = Vector CoV2Ngene-Luc2-ZsGreen
  source 1..9501: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaataaaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaaggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 13 - DNA, 1260 bp, synthetic construct
  misc_feature 1..1260: note = Inserted N gene sequence
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 14 - DNA, 5822 bp, synthetic construct
  misc_feature 1..5822: note = Vector VSV-G
  source 1..5822: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

(19) United States
(12) Patent Application Publication
(10) Pub. No.: US 2025/0180560 A1
(43) Pub. Date: Jun. 5, 2025

(54) Method for Rapid Capture, Detection and Analysis of Viruses on Surfaces

(71) Applicant: Northeastern University, Boston, MA (US)

(72) Inventors: Sara H. Rouhanifard, Chestnut Hill, MA (US); Yifang Liu, Framingham, MA (US); Jacob Lemieux Potts, Brookline, MA (US); Dylan Joshua Goldbloom Bloch, Needham, MA (US)

(21) Appl. No.: 18/844,800

(22) PCT Filed: Mar. 9, 2023
(86) PCT No.: PCT/US2023/064078

§ 371 Date: Sep. 6, 2024

Related U.S. Application Data
(60) Provisional application No. 63/269,062, filed on Mar. 9, 2022.

(51) Int. Cl. G01N 33/569; C12Q 1/6841; G01N 33/53

(52) U.S. Cl. CPC G01N 33/56983; C12Q 1/6841; G01N 33/5308; G01N 2333/155; G01N 2333/165

(57) ABSTRACT

Provided herein are, in various embodiments, methods and kits for assaying one or more virions. In certain embodiments, the methods and kits of the disclosure provide for the calculation of virion titer and/or virion infectivity. In still further embodiments, the disclosure provides for methods and kits for enhancing assaying of viruses such as SARS-CoV-2.

### RELATED APPLICATION

This application claims the benefit of U.S. Provisional Application No. 63/269,062, filed on Mar. 9, 2022. The entire teachings of the above application is incorporated herein by reference.

### GOVERNMENT SUPPORT

This invention was made with government support under 2141135 and 2032533 from the National Science Foundation. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 52002294002.xml; created Mar. 9, 2023, 77,824 Bytes in size.

### BACKGROUND

Rapid and accurate virus concentration measurement is desirable in both diagnostic and research and development settings. Clinically, rapid detection of infectious viral particles enables a timely diagnosis, and in industry, viral vectors are a significant component of gene therapies, vector-based vaccines, and CAR-T immunotherapies. Currently, the most common methods for quantifying viruses are either based on the enzyme-linked immunosorbent assay (ELISA)-based detection of viral surface antigen, which is rapid but not sensitive, or PCR-based detection of the viral genome, which is sensitive but not rapid. A shortcoming of each of these assays is that the presence of either surface antigen or viral genome does not necessarily indicate the concentration of infectious viral particles.

Accordingly, there exists a need in the art for a direct, fast, and sensitive assay for virus detection.

### SUMMARY

In one aspect, the present disclosure provides a method of assaying a virion comprising, capturing the virion on a solid substrate, fixing the virion on the solid substrate, washing the solid substrate, and detecting the virion. In one aspect, the present disclosure provides for a method of assay a virion wherein virion was obtained from a cell-free aliquot. In some embodiments, the virion is a cell-free aliquot. In some embodiments, the virion is obtained from cells and transferred to a cell-free solid substrate.

In one aspect, the disclosure provides for a solid substrate, e.g., wherein the solid substrate is glass or plastic, e.g., a polystyrene plate or a cover glass (as used herein, “coverglass” and “cover glass” are equivalent). In one aspect, the present disclosure provides for a step of capturing the virion on a solid substrate comprising, coating the solid substrate, immobilizing a binder to the solid substrate, and contacting the solid substrate with the virion, thereby capturing the virion on the solid substrate. In one aspect, the present disclosure provides for a step of capturing the virion on a solid substrate comprising, coating the solid substrate with streptavidin, immobilizing a biotinylated binder to the streptavidin-coated solid substrate, and contacting the solid substrate with the virion, thereby capturing the virion on the solid substrate.

In one embodiment, the solid substrate is coated with streptadivin. In another embodiment, the solid substrate is coated with cross linker(s).

In one embodiment, the binder is biotinylated. In one aspect, the binder is an aptamer, a nanoparticle, a lectin, a protein, or a chemical binder. In one aspect, the binder is an aptamer comprising from about 10 to about 60 nucleotides.

In one aspect, the disclosure provides for an aptamer further comprising a salt, e.g., to enable the aptamer to fold properly and bind. In one aspect, the salt is a magnesium salt, e.g., a magnesium chloride. In one aspect, the salt is a chloride salt. In one aspect, the present disclosure provides for a step of fixing the virion on the solid substrate. In one aspect, fixation is methanol fixation.

In further aspects, the disclosure provides for calculating virion titer. In one aspect, the disclosure provides for the quantification of virion infectivity. In one aspect of the disclosure, the method is used on more than one virion type. The virion can be any virus (e.g., a DNA virus, an RNA virus, etc.). In one aspect, the virion is SARS CoV-2, Varicella-zoster, lentivirus, adenovirus, or any combination thereof.

In one aspect, the disclosure provides for a kit for assaying at least one virion in a sample. In one aspect, the kit comprises a solid substrate, a fixation solution, and a wash solution. In one aspect, the kit is adapted for storage at room temperature.

In one aspect, the methods described herein are nucleic acid-based, e.g., DNA-based or RNA-based. In one aspect, the methods are DNA-based. In one aspects, the method are RNA-based.

In one aspect of the methods described herein, at least two types of virions are assayed. In one aspect, the methods further comprise assaying at least two types of virions.

In one aspect of the methods described herein a ratio of at least two types of virions is determined. In one aspect, the methods further comprise determining a ratio of the at least two types of virions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B aptamers can capture recombinant spike protein. FIG. 1A shows aptamers modified with 5′ or 3′ biotin (left) or ACE2 proteins with biotin (right) were conjugated to a streptavidin-coated polystyrene plate. Following spike binding (tulip), the spike protein was detected with an anti-spike antibody conjugated to horseradish peroxidase (HRP) (star), followed by colorimetric detection.

FIG. 1B shows measurements for the binding specificity of 1 C and 4 C aptamers. Different conditions of aptamers were tested (5′ modified, 3′ modified, ±MgCl₂). n=3 biological replicates (mean±SD). Plates without a biotinylated binder (none) were set as a baseline to show a significant difference from other groups. An unpaired t-test was performed to compare samples. *p<0.05, **p<0.01, ***p<0.001, ****p<0.0001.

FIG. 2 shows RT-qPCR quantification of lentiviral vectors used in this study. Multiple virus strains were quantified after production, genome copies number/μL are shown, no RT means no reverse transcription was performed, S+ represents presence of spike protein on the virus surface, N+ represents presence the Nucleocapsid gene in the lentiviral genome, S-represents absence of any viral coat protein, and V+ represents presence of Vesicular Stomatitis Virus Glycoprotein (VSVG) on the virus surface. n=2 biological replicates (bars represent mean±SD).

FIGS. 3A and 3B aptamers can capture viruses via spike protein. FIG. 3A shows RT-qPCR for capture efficiencies of SARS-CoV-2 spike pseudotyped lentivirus by 1 C aptamers. 1 C aptamers modified with 5′ biotin was conjugated to a streptavidin-coated polystyrene plate. Following lentivirus capture by the aptamers, RNA was extracted, and RT-qPCR was performed to measure the number of captured virions.

FIG. 3B shows a bar graph of genome copies number per mL of spike and VSVG virions captured by 1 C aptamers and random aptamers. n=3 biological replicates (mean±SD). *p<0.05, **p<0.01, ***p<0.001, ****p<0.0001.

FIG. 4A shows RT-qPCR for capture efficiencies of SARS-CoV-2 spike pseudotyped lentivirus by ACE2 receptor. ACE2 receptors were conjugated to a streptavidin-coated polystyrene plate. Following lentivirus were captured by the receptors, RNA was extracted, and RT-qPCR was performed to measure the amount of captured virions.

FIG. 4B shows a bar graph of genome copies number per mL of spike and VSVG virions captured by ACE2 receptors and random aptamers. n=3 biological replicates (mean±SD). *p<0.05, **p<0.01, ***p<0.001, ****p<0.0001.

FIG. 4C shows saturation test of 1 C aptamers concentration. Six different concentrations of 1 C aptamers ranging from 1 μM to 200 μM were tested, VSVG lentivirus was used as a negative control to indicate nonspecific binding to 1 C aptamer, the genome copy number per mL of spike and VSVG virions captured by difference concentration of 1 C aptamers were shown in the bar graph. n=3 biological replicates (mean±SD).

FIG. 5A shows RT-qPCR for capture efficiencies of SARS-CoV-2 spike pseudotyped lentivirus by 1 C aptamers on chambered coverglass. To prove virions can be captured on coverglass applicable for FISH, chambered coverglass was biotinylated by oxygen plasma, then streptavidin was added, and biotinylated 1 C aptamer was immobilized. The spike-pseudotyped lentivirus and the VSVG-pseudotyped lentivirus were added to the coverglass at concentrations of 106 genome copies per μl. After washing, we extracted the RNA from captured virions and performed RT-qPCR targeting the CMV promoter (common between both lentivirus vectors) to determine capture efficiency. No streptavidin-treated sample was used as the negative control.

FIG. 5B shows a bar graph of genome copies per mL of spike and VSVG virions captured by 1 C aptamers and random aptamers. n=3 biological replicates (mean±SD). *p<0.05, **p<0.01, ***p<0.001, ****p<0.0001.

FIG. 6 shows an example embodiment of a workflow of raptamer FISH detection. In the image, the arrow to the left of “Methanol fixation” extends from “virus capture.” Other example embodiments use agents other than streptavidin and/or a biotinylated binder.

FIG. 7 shows DNA agarose gel electrophoresis to prove the integrity of viral genome post-methanol fixation and post-TurboFISH treatment. RT+ means reverse transcription was performed. Virions were captured, and viral mRNA was extracted post methanol or TurboFISH, RT-PCR targeting the N gene of S+N+ was performed to detect the 1260 bp band in different binding reagents.

FIGS. 8A-8D raptamer FISH detection of individual virions. FIG. 8A shows raptamer FISH using ACE2 receptor.

FIG. 8B shows raptamer FISH using 1 C aptamers.

FIG. 8C shows fluorescent micrographs showing raptamer FISH using ACE2 receptor targeting N gene of the viral genome colocalized FISH spots per 20000 μm² were quantified in bar graphs by the Matlab colocalization analysis pipeline. 3 biological replicates, 5 images from each replicate (mean±SD). The overlay image shows colocalized spot (“singlet”) and colocalized spot (clustered).

FIG. 8D shows fluorescent micrographs showing raptamer FISH using 1 C aptamers targeting N gene of the viral genome colocalized FISH spots per 20000 μm² were quantified in bar graphs by the Matlab colocalization analysis pipeline. 3 biological replicates, 5 images from each replicate (mean±SD).

FIG. 9A shows particle size (pixel) distribution of FISH spots in FIG. 8. After the binarization step, the particles detected in the alexa 488 channel were measured in Matlab, histogram of size distribution was shown. 1 C aptamer group capture spike coated lentivirus with N gene was compared to no 1 C aptamer group capture spike coated lentivirus without N gene.

FIG. 9B shows percent of colocalized spots for small spots (<70 pixels) and large spots (>70 pixels) in 1 C captured spike coated lentivirus with N gene. n=3 biological replicates (mean±SD).

FIG. 10A shows multiplexed virus detection by TurboFISH. Multiple virus strains with different genomes were detected by N gene probes and luciferase probes with two sets of dyes.

FIG. 10B shows that different amount ratios of two virions were detected to show whether the FISH spots ratio of two targets matches the concentration ratio of two virions added. 3 biological replicates, 5 images from each replicate (mean±SD).

FIGS. 11A-11E Aptamers enrich for infectious virions. FIG. 11A shows functional titering of uncaptured virions from aptamers and ACE2 to prove the infectivity of captured virions. a. Uncaptured supernatant virions were aspirated out and used to infect cells. Flow cytometry was performed to count the percentage of infected cells.

FIG. 11B shows images of cells infected by uncaptured virions from different binding reagents. FIG. 11B-1 is the cell nucleus was stained with DAPI. FIG. 11B-2 is zsgreen expression indicating viral infection.

FIG. 11C shows histogram plots of flow cytometry data, uninfected/infected cells were gated based on viral infection of 293 FT cells. The ACE2 group was compared to the uncoated group, and the 1 C aptamer group was compared to the random aptamer group.

FIG. 11D shows normalization of uncaptured virions infectivity, percent of infected cells in different binding reagents was normalized to the uncoated group. *p<0.05, **p<0.01, ***p<0.001, ****p<0.0001.

FIG. 11E shows fold decrease of TU/mL of ACE2 and 1 C aptamer groups were compared to the uncoated group. n=5 biological replicates (mean±SD). *p<0.05, **p<0.01, ***p<0.001, ****p<0.0001.

FIG. 12 shows brightfield images of monolayer cells in FIGS. 11B-1 and 11B-2.

FIG. 13A shows correlation of raptamer FISH detection with functional titering. FIG. 13A-1 shows images of infected ACE2 expressing HEK293FT cells (zsgreen expression indicating viral infection; top), and images of colocalized FISH spots from different concentrations of virions (bottom). FIG. 13A-2 shows cell nuclei stained with DAPI.

FIG. 13B shows quantification of infectious virions by comparison of colocalized FISH spots (y axis) with cell transducing units (x axis). Points are showing the mean of two biological replicates.

FIG. 14 shows brightfield images of monolayer cells in FIG. 13A.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Early detection of viruses can prevent the uncontrolled spread of viral infections. Determination of viral infectivity is also critical for determining the dosage of gene therapies, including, for example, vector-based vaccines, CAR T-cell therapies, and CRISPR therapeutics. In both cases, for viral pathogens and viral vector delivery vehicles, fast and accurate measurement of infectious titer is desirable. The most common methods for virus detection are antigen-based (rapid but not sensitive) and reverse transcription polymerase chain reaction (RT-PCR)-based (sensitive but not rapid). Current viral titer methods heavily rely on cultured cells, which introduces variability, e.g., within labs and between labs. Thus, it is highly desirable to directly determine the infectious titer without using cells.

Sensitive methods for viral detection (i.e., PCR) require the use of enzymes that require refrigeration and time to achieve detectable levels due to extraction and amplification of the viral genome. These methods also require significant reagents for RNA extraction and processing that can be significantly affected by supply chain issues, such as those occurring during the Covid-19 pandemic. Alternatively, rapid antigen detection methods are faster but lack the sensitivity to detect low levels of virus. There is significant need for methods that are fast and sensitive, avoid refrigeration and/or heating, and circumvent supply chain issues that are being faced globally. Rapid detection with high sensitivity and specificity may be achieved to detect the viral genome, however, these methods do not work on individual virions and require detection within infected cells. In embodiments, the present disclosure provides methods and kits to detect individual virions, rapidly and with high sensitivity and selectivity, and, in some aspects, without the use of temperature-sensitive or specialized reagents (circumventing possible supply chain issues).

Here, in one embodiment, the present disclosure provides for the development of a direct, fast, and sensitive assay for virus detection (dubbed rapid-aptamer FISH or raptamer FISH) and cell-free determination of infectious titers. Raptamer FiSH refers to turboFISH where the virus is captured by an aptamer. The present disclosure demonstrates that the virions captured are “infectious,” thus serving as a more consistent proxy of infectious titer. This example assay first captures viruses bearing an intact coat protein using an aptamer, then detects genomes directly in individual virions using fluorescence in situ hybridization (FISH)—thus, it is selective for infectious particles (i.e., positive for coat protein and positive for genome).

The infectious titer of a virus is often calculated in transducing units (TU) per mL. The total particle-to-TU ratio is the total number of particles divided by the TUs. For many viruses, the particle-to-TU ratio can be extremely high and variable. For example, the ratio for Varicella-zoster virus is 40,000:1, while for adenovirus (HAdV-C5), it is 58:1. For HIV-1, the ratios have even more variability ranging from 1-10⁷:1. In these cases, the properties measured biochemically may not be directly attributed to those of the infectious particles, thus complicating the quality control and assessment of performance for gene therapies in vivo and evaluation of how infectious an individual is at a given moment.

The surface antigen test (ELISA) may detect viruses with surface antigens but no genome. By contrast, PCR-based tests may detect viruses that contain a genome but no surface antigens. For example, the viral RNA of SARS-CoV-2 patients can still be detected even in the absence of an infectious virus. Functional titering of infectious virions is accomplished with a viral plaque assay or tissue culture infectious dose −50 (TCID50), both cell-based. These methods titer virions by measuring the cytopathology in cell monolayers produced by viral replication. However, functional titering using cell culture can lead to significant variability from cell type, passage number, and condition of the cells. Therefore, a cell-free titering method is needed and can improve the accuracy and consistency of viral quantification.

Single-molecule RNA FISH (smFISH) is a sensitive method that directly detects RNA. It uses approximately 30 fluorescently labeled oligonucleotide probes to target RNA sequences. The tiling of probes across an RNA sequence amplifies the fluorescent signal locally and enables the direct detection of individual RNA molecules using fluorescence microscopy. SmFISH is frequently used to detect virus-infected cells and tissues. It has also been reported to detect individual virions via the capture of surface antigens; however, these methods required specialized instrumentation (i.e., Raman spectroscopy, TIRF microscopy). Two alternative smFISH methods, TurboFISH and rvFISH can decrease the total time of the assay from 12 hours to <20 minutes while maintaining the sensitivity and specificity of the assay, making these methods ideal candidates for rapid virus detection. However, TurboFISH detects viral genomes in infected cells, and rvFISH detects all viral particles containing the genome, irrespective of infectivity.

Accordingly, in one aspect, the present disclosure provides for a rapid assay to directly detect intact virions (dubbed rapid-aptamer FISH or “raptamer FISH”) using epifluorescence microscopy for targeting both viral genomes and coat protein, thus providing a better proxy for the overall infectivity of the detected virus. In some aspects, the present disclosure applies both antibodies and aptamers that are specific for viral coat proteins to capture virions directly onto a solid substrate, e.g., glass. Once captured, viral genomes were detected by TurboFISH to inform the infectivity of virions (positive for coat protein and positive for genome). The use of two probe sets targeting different genomic regions increases the specificity of viral RNA detection. Thus, the present disclosure demonstrates that infectious virions are bound to the antibodies and aptamers by performing a functional titer on the unbound fraction of the virus and showing a marked decrease in infectivity. Overall, these results demonstrate that raptamer FISH can replace cell-based assays for determining infectivity, providing a useful and fast readout for manufacturers of gene therapies that are delivered via viral vectors, as well as a useful and fast measurement of the infectivity of an individual.

### Methods of Assaying

In one aspect, the present disclosure provides a method of assaying a virion comprising, capturing the virion on a solid substrate, fixing the virion on the solid substrate, washing the solid substrate, and detecting the virion.

As used herein, the term “virion” refers to any complete virus particle that consists of an RNA or DNA core with a protein coat and optionally with at least one external envelope.

In one aspect, the present disclosure provides for a method of assay a virion wherein the virion is a cell-free aliquot. In one aspect, the virion is obtained from one or more cells and then transferred to a cell-free solid substrate. As used herein, the term “cell-free” is used to define a system, a process, or a platform where biochemical reactions occur independently of living cells (e.g., yeast cells, bacterial cells, etc.). In one aspect, the assay is a non-cell-based assay.

In one aspect, the disclosure provides for a solid substrate wherein the solid substrate is a polystyrene plate or glass, e.g., a cover glass.

In one aspect, the present disclosure provides for a step of capturing the virion on a solid substrate comprising, coating the solid substrate with, e.g., a streptavidin or chemical crosslinker, immobilizing a binder to the coated solid substrate, wherein the binder is a biotinylated binder or chemical binder, and contacting the solid substrate with the virion, thereby capturing the virion on the solid substrate. In one aspect, the chemical crosslinker is a maeleimide-thiol, disulfide, or azide-alkyne.

In one aspect, the binder is an aptamer, a nanoparticle, a lectin, a protein, or a chemical binder. In one aspect, the binder is a maeleimide-thiol, disulfide, or azide-alkyne. In one aspect, the biotinylated binder is an aptamer comprising from about 10 to about 60 nucleotides, for example, about 5-15, 5-20, 10-15, 10-20, 15-20, 20-23, 18-22, 15-25, 20-25, 20-30, 25-40, 30-45, 35-50, 45-60, or 20-60 nucleotides. In one aspect, the aptamer is at least about 20 nucleotides. In one aspect, the binder comprises a series of oligonucleotides, wherein each nucleotide comprises from about 10 to about 60 nucleotides (e.g., about 20 nucleotides). In one aspect, the aptamer is a DNA aptamer. In one aspect, the aptamer is an RNA aptamer. In one aspect, the aptamer is serum-stable. In one aspect, the aptamer is specific, e.g., specific for SARS-CoV-2. In one aspect, the aptamer targets the Receptor-Binding Domain of a spike protein, e.g., the SARS-CoV-2 spike glycoprotein. In one aspect, the aptamer is set forth in Valero, Julián et al. “A serum-stable RNA aptamer specific for SARS-CoV-2 neutralizes viral entry.” Proceedings of the National Academy of Sciences 118.50 (2021): e2112942118 and/or Song, Yanling et al., “Discovery of aptamers targeting the receptor-binding domain of the SARS-CoV-2 spike glycoprotein.” Analytical chemistry 92.14 (2020): 9895-9900 (the contents of which are herein incorporated by reference in their entirety).

In one aspect, the disclosure provides for an aptamer further comprising a salt, e.g., to enable the aptamer to fold properly and bind. In one aspect, the salt is magnesium salt. In one aspect, the salt is magnesium chloride.

In alternative embodiments of methods described herein, the substrate is covered with sulfurs. In some embodiments, the capture is based on charge (for example, where the surface is coated with a positively-charged coating and the virus is negatively charged). In some embodiments, a lectin-capturing sugar is used. In some embodiments, the substrate is contacted with the virus, and the virus is allowed to dry. In some embodiments, RNAscope is used. See Wang F, Flanagan J, Su N, Wang L C, Bui S, Nielson A, Wu X, Vo H T, Ma X J, & Luo Y (2012), RNAscope: a novel in situ RNA analysis platform for formalin-fixed, paraffin-embedded tissues. The Journal of molecular diagnostics: JMD, 14 (1), 22-9 PMID: 22166544.

In one aspect, the present disclosure provides for a step of fixing the virion on the solid substrate. In one aspect, fixation is methanol fixation or crosslinking. In one aspect, the crosslinking is formaldehyde or glutaraldehyde.

In one aspect, the methods comprise imaging. In one aspect, the present disclosure provides for imaging modalities such as, for example, epifluorescence microscopy and/or fluorescence in situ hybridization (FISH). In some embodiments of the disclosure, the modality is TurboFISH as described in the Shaffer, S. M.; Wu, M. T.; Levesque, M. J.; Raj, A. Turbo FISH: A Method for Rapid Single Molecule RNA FISH. PLoS One 2013, 8 (9), e75120 (the contents of which are herein incorporated by reference in their entirety). In other aspects, the FISH is single molecule FISH (smFISH), HCR-FISH, bDNA-FISH, ClampFISH, or other FISH derivative or modality.

In some aspects of the method, a permeabilizing agent is used. In some aspects, hybridization is used for amplification.

In some embodiments, viral detection and identification comprise methods described in Liu, Y. et al. Paired aptamer capture and FISH detection of individual virions enables cell-free determination of infectious titer. bioRxiv 2022.11.13.516306 (2022) doi: 10.1101/2022.11.13.516306, along with the Supporting Information, Supplementary methods and Supplementary Note: Sequences, all incorporated herein by reference in entirety.

In some embodiments, viral detection and identification comprise methods described in Hepp, C.; Shiaelis, N.; Robb, N. C.; Vaughan, A.; Matthews, P. C.; Stoesser, N.; Crook, D.; Kapanidis, A. N. Viral Detection and Identification in 20 Min by Rapid Single-Particle Fluorescence in-Situ Hybridization of Viral RNA. Sci. Rep. 2021, 11 (1), 19579, incorporated by reference in its entirety.

In one aspect, the disclosure provides for probes, a probe set, or probe sets. As used herein, a “probe set” means a collection of probes that are designed to hybridize to a desired DNA or RNA target sequence. In at least one aspect, the probe set comprises probe sets, e.g., probe sets in different fluorescent channels. In one aspect, the probes or probe sets are optimized.

In one aspect, the disclosure provides for calculating virion titer. In one aspect, calculating virion titer comprises binarization, colocalization, and size thresholding. In some aspects, one or all of the steps of binarization, colocalization, and/or size thresholding may be performed by image processing software. In some aspects, the steps are performed sequentially and/or iteratively. In one aspect, the disclosure provides for the quantification of virion infectivity. As used herein, “viral infectivity” is defined as the number of virus particles capable of invading a host cell.

In one aspect, image processing selects for size (to exclude debris and virus aggregates), probe colocalization for specificity of detection, and uses multiple probes to discriminate different viruses in the sample.

In one aspect of the disclosure, the method is used on more than one virion type. In one aspect, the virion is SARS CoV-2, Varicella-zoster, lentivirus, adenovirus, or any combination thereof.

In one embodiment, the virion is from a sample, e.g., a biological sample, such as bodily fluid from a human (e.g., subject or patient) or from an animal (such as a mammal) which is not human. In some aspects, the sample is a saliva sample or a breath sample. In some aspects, the sample is exhaled breath. In some aspects, the sample is saliva. In some aspects, the samples are collected by harvesting saliva or to collecting exhaled breath, and detecting individual virions. In some embodiments, the method involves use of a solid substrate that had been breathed on by a subject. In some embodiments, the sample is mucus, spinal fluid, or plasma. In some embodiments, the sample is from water containing sewage. In some embodiments, the bodily sample is a solid taken from the subject, e.g., a solid tumor or stool sample, to which liquid is added. In some embodiments, the sample comprises viruses, microbes, bacteria, fungi, lipid nanoparticles comprising DNA or RNA, bacteriophage, or any combination thereof. In some embodiments, multiple viruses or strains in a single sample are detected, e.g., using different aptamers and using labels. In some embodiments, a naked virus is detected.

In some embodiments, the methods are used to capture viruses for ELISA assays.

In some embodiments, aptamers may be selected via SELEX for any surface antigen, including aptamers that target the spike protein for SARS CoV-236, and RNA FISH probes may be designed to target different viral strains. See Kohlberger, M.; Gadermaier, G. SELEX: Critical Factors and Optimization Strategies for Successful Aptamer Selection. Biotechnol. Appl. Biochem. 2021. https://doi.org/10.1002/bab 2244, incorporated by reference in its entirety. These properties allow the system to be rapidly customized and applied to emerging pathogens and any desired viral vector without the need for fluorescent transgenes that are either not present in natural pathogens or not permitted in the minimum payload for gene therapies.

In some embodiments, the disclosure provides a direct, fast, and sensitive assay for virus detection and cell-free determination of infectious titers with no thermal requirement. In some embodiments, the assay first captures viruses bearing an intact coat protein using an aptamer, then detects genomes directly in individual virions using FISH-thus selecting for “infectious” particles. The virions captured are “infectious,” thus serving as a proxy of infectious titer. This measurement circumvents the need for cell-based assays to determine infectious titers for diagnostic and biotechnology settings.

In one aspect, the disclosure provides for methods and kits used for cell-free determination of infectious titer.

In one aspect, the disclosure provides for methods and kits used for capturing infectious particles.

In one aspect, the disclosure provides for a kit for assaying at least one virion in a sample. In one aspect, the kit comprises a solid substrate, a fixation solution, and a wash solution. In one aspect, the kit is adapted for storage at room temperature. In one aspect, the solid substrate is a polystyrene plate or a cover glass. In one aspect, the solid substrate has an upper surface and a lower surface, wherein the upper surface is coated with streptavidin. In one aspect, a biotinylated binder is bound to the streptavidin-coated upper surface of the solid substrate. In one aspect, the biotinylated binder is an aptamer, a nanoparticle, a lectin, a protein, or a chemical binder. In one aspect, the biotinylated binder is an aptamer comprising from about 10 to about 60 nucleotides. In one aspect, the aptamer is at least about 20 nucleotides. In one aspect, he fixation solution is methanol. In one aspect the kit is for use in any of the methods described herein. In one aspect of the invention, the method comprises use of a kit, for example, a kit described herein.

In some aspects of the invention, viral particles may be captured directly on surfaces using specific DNA sequences that bind to viral coat proteins; in some aspects, viral particles may be captured rapidly on surfaces (e.g., in as little as 1 minute); and in some aspects. positive identification of viral genome may be achieved rapidly (e.g., in as little as 10 minutes) and with high sensitivity and specificity.

In one example aspect, a two-part, rapid method is used to capture viral particles (specifically SARS CoV-2, but it can be applied to any virus) and directly detect them on surfaces. The first part of the method is based on the binding properties of DNA aptamers that bind specifically to viral surface proteins (i.e., spike protein) for affinity capture onto different surfaces (e.g., glass). DNA aptamers are highly specific towards their target and are made of short DNA sequences, so they are thermostable and easily synthesized and quality controlled, ideal conditions for a widely deployable tool for detecting viral infections. Further, they can reduce batch-to-batch variability. Once the viruses are immobilized by the aptamer, a single-molecule RNA fluorescence in situ hybridization (FISH) is applied to detect single molecules of RNA in individual virions, e.g., in under 10 minutes. The set-up is a glass cover slide that has the DNA aptamer chemically conjugated to the surface and dried down in the presence of magnesium. This dry surface is where the sample (e.g., virus infected breath or saliva) will be added. The fluid in the sample will rehydrate the aptamer and dissolve the magnesium into solution, aiding in proper aptamer folding and subsequent binding to the viral surface protein. After the viral particles have been captured, they will be subject to fixation and permeabilization, optimized to form a scaffold so the RNA does not escape the permeabilized virion but porous enough to allow entry of detection probes and exit of unbound probes during washes. In the last step, virions are hybridized using 20mer DNA oligonucleotide probes that are complementary to the target RNA sequence in 2 different fluorescent channels to ensure specific fluorescent detection. This hybridization is performed with a specific combination of surfactants and blocking agents to eliminate non-specific binding and maximize specific probe binding and total fluorescent signal produced from each virion.

Example workflow for an aspect of the disclosure:
1. Prepare and store dry, glass cover slides with aptamer and magnesium covalently attached.
2. Rehydrate aptamer and magnesium on slide with viral sample while binding to glass.
3. Fix and permeabilize captured virions on glass surface.
4. Hybridize multiple probe sets (minimum of 2) targeting different regions of viral genome using different fluorophores in the presence of multiple blocking agents to eliminate background fluorescence binding. Colocalization of signal indicates positive viral genome detection.

Positive signal indicates detection of viral genome as well as viral surface protein. This implies that an intact viral particle is being detected rather than remnant of viral genome after a viral infection has passed that may be detected using PCR-based methods.

### Methods

### Tissue Culture Conditions

HEK293FT cells (ThermoFisher cat #R700-07) were handled according to the manufacturer's protocol and maintained in high glucose DMEM with GlutaMax (FisherSci cat #10566016), 10% FBS, 1% MEM Non-Essential Amino Acids (ThermoFisher cat #R70007), 1% supplementary GlutaMax (FisherSci cat #35-050-061), 1% MEM sodium pyruvate (ThermoFisher cat #11360070), 1% Pen-Strep (FisherSci cat #BW17-602E), and 500 μg/mL geneticin (ThermoFisher cat #10131035). HEK293T-ACE2 cells described in Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5) (the contents of which are herein incorporated by reference in their entirety) (BEI cat #NR-52511) were cultured in the HEK293FT media without geneticin. Cell lines tested PCR-negative for mycoplasma contamination (ATCC cat #30-1012K).

### Plasmids

The following plasmids were ordered as part of a lentiviral kit (BEI cat #NR-52948) described in Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5) (the contents of which are herein incorporated by reference in their entirety): Spike (BEI cat #NR-52514), Luc2-ZsGreen (BEI cat #NR-52516), Hgpm2 (BEI cat #NR-52517), Tat1b (BEI cat #NR-52518), and Rev1b (BEI cat #NR-52519). VSV-G plasmid (Addgene cat #12259) was a gift from the lab of Jiahe Li. To prepare the Spike G614 Δ19 plasmid, we generated 2 PCR amplicons using mutagenic primers, Q5 polymerase (NEB cat #M0491) and Spike plasmid (BEI cat #NR-52514) as a template. PCR reactions were digested in DpnI, and the amplicons were joined via Gibson assembly (NEB cat #E2611). The coding sequences of generated plasmids were sequence-verified. To prepare the N gene inserted plasmids (CoV2Ngene-Luc2-ZsGreen) to model SARS-CoV-2, we removed an 1131 bp segment of luciferase in Luc2-ZsGreen (BEI cat #NR-52516), and the N gene (1260 bp, IDT 2019-nCOV_N_Positive Control Plasmid cat #10006625) was amplified by mutagenic primers. The remaining part of Luc2-ZsGreen (BEI cat #NR-52516) and the amplified N gene were ligated via Gibson assembly (NEB cat #E2611). The coding sequences of generated plasmids were sequence-verified.

### Generating Pseudotyped Lentiviral Particles

The protocol follows the previously described in Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5) (the contents of which are herein incorporated by reference in their entirety), with several modifications. HEK293FT cells were seeded in media without geneticin at a density such that cells were 50% confluent the next day. 16 to 24 hours after seeding, cells were transfected with the lentiviral plasmids using BioT (Bioland cat #B01-01) according to the manufacturer's protocol. The number of plasmids per transfection was at a 1:2/1:9/1:6 mass ratio for the lentiviral backbone (Luc2-ZsGreen or N-ZsGreen), helper plasmids (Hgpm2, Tat1b, and Rev1b), and viral entry protein (Spike G614 Δ19, or VSV-G), respectively. Media was changed 18 hours post-transfection (hpt). Viral supernatant was collected at 60 hpt and kept and centrifuged at 500×g for 10 min to clear cell debris. Cleared supernatant was immediately frozen at −80° C. in aliquots.

### Viral Quantification

Viruses were quantified via a RT-qPCR method described in Zhang, L.; Jackson, C. B.; Mou, H.; Ojha, A.; Peng, H.; Quinlan, B. D.; Rangarajan, E. S.; Pan, A.; Vanderheiden, A.; Suthar, M. S.; Li, W.; Izard, T.; Rader, C.; Farzan, M.; Choe, H. SARS-CoV-2 Spike-Protein D614G Mutation Increases Virion Spike Density and Infectivity. Nat. Commun. 2020, 11 (1), 6013 (the contents of which are herein incorporated by reference in their entirety), where the only deviation were using different DNase I (ThermoFisher #AM1907) digestion, reverse transcriptase (FisherSci #18-080-044), and plasmid (Luc2-ZsGreen) for generating standard curves. A CFX96™ Optics Module, C1000 Touch™ Thermal Cycler (BIO-RAD). Finally, viruses were functionally titered via HEK293T-ACE2 infection as previously described in Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5) (the contents of which are herein incorporated by reference in their entirety), with the only deviation being that the HEK293T-ACE2 cells were cultured in HEK293FT media without geneticin. 48 h post-infection, cells were analyzed with the Attune NxT flow cytometer for fluorescence, indicating a successful viral infection. The transducing units of infectious virions were obtained from the P (percent of infective positive cells) based on the formula:

$$TU / mL = - ln ( 1 - [ P / 100 ] ) ⋆ ( \# cells in well / volume of viral stock added to well in mL )$$

### Chemical Immobilization of Biotin to a Chamber Coverglass

To generate the biotinylated coverglass necessary to run and image raptamer FISH, an 8-well chambered coverglass (ThermoFisher #155409) was conditioned in an oxygen-plasma cleaner to introduce the active oxygen group to the slide. The slide was cleaned with water and then 100% isopropanol before the reaction. The slide was treated by oxygen at 300 m Torr pressure for 1 min and then by plasma power at 50 mW for 1 min. After the oxygen plasma, silane-PEG-biotin (10 mg/mL) dissolved in 99% ethanol was added to react with the oxygen group and incubated for 4-6 h at RT. The slide was washed with 99% ethanol three times after silane-PEG-biotin treatment. The dried slide was kept at 4° C. overnight for use the next day.

### ELISA Assay

Recombinant spike protein (BPS bioscience #100810) was captured by ACE2 receptor protein (Acro biosystems #AC2-H82F9) and aptamers (CoV2-RBD-1C and CoV-RBD-4C) targeting receptor-binding domains of SARS-CoV-2 spike glycoprotein which were discovered in ong, Y.; Song, J.; Wei, X.; Huang, M.; Sun, M.; Zhu, L.; Lin, B.; Shen, H.; Zhu, Z.; Yang, C. Discovery of Aptamers Targeting Receptor-Binding Domain of the SARS-CoV-2 Spike Glycoprotein (the contents of which are herein incorporated by reference in their entirety). Biotin-modified aptamers and ACE2 were bound to streptavidin-coated polystyrene plates (ThermoFisher #PI15500). The aptamers and ACE2 captured the recombinant spike protein, and then SARS-CoV-2 Spike Protein Monoclonal Antibody [H6] HRP-conjugated (AssayGenie #PACOV0004) was used to detect the captured spike protein. The ELISA procedure to detect spike protein was conducted according to the streptavidin-coated plate manufacturer's protocol with the following changes: Aptamers (10 μM), spike protein (1 ug/mL), and monoclonal antibodies (1 ug/mL) were dissolved in the binding buffer (1× PBS, 0.55 mM MgCl₂). ACE2 protein (500 ng/mL) was dissolved in the wash buffer (25 mM Tris, 150 mM NaCl, 0.1% FBS, 0.05% Tween-20). The wells were pre-washed with 1×PBS for 10 minutes and then the binding buffer for 10 minutes before adding the aptamers. Aptamers were heated at 90° C. for 10 minutes and placed on ice for 10 minutes. The aptamers and ACE2 protein had 2 hours of binding time. Both the spike protein and the antibody had 30 minutes of binding time. The plates were pre-incubated with HEK293T-ACE2 cell media with 0.55 mM MgCl₂ for 10 min. The virus was prepared in HEK293T-ACE2 cell media with 0.55 mM MgCl₂ and ssDNA. The virus was added to the plates and incubated for 45 min, and the plates were washed 3 times with binding buffer after virus incubation. For biotinylated coverglass, streptavidin protein (100 μg/mL) was added first and incubated for 1 h, and the remaining steps were the same as described above.

### RNA Extraction, RT-PCR and RT-qPCR

Methods were adapted from Zhang, L.; Jackson, C. B.; Mou, H.; Ojha, A.; Peng, H.; Quinlan, B. D.; Rangarajan, E. S.; Pan, A.; Vanderheiden, A.; Suthar, M. S.; Li, W.; Izard, T.; Rader, C.; Farzan, M.; Choe, H. SARS-CoV-2 Spike-Protein D614G Mutation Increases Virion Spike Density and Infectivity. Nat. Commun. 2020, 11 (1), 6013 (the contents of which are herein incorporated by reference in their entirety). Frozen viral aliquots were thawed and treated with RNase A (EN0531 ThermoFisher, diluted 10×) for 1 h at 37° C. to degrade RNAs that were not packaged inside the virion. RNA was extracted using Trizol (ThermoFisher #15596026) and resuspended in NF water. DNase (ThermoFisher #AM1907) was used to remove the contaminant DNA. RNA was reverse transcribed (FisherSci #18-080-044). The Luna qPCR (NEB #M3004) was used to quantify the generated cDNA. OneTaq RT-PCR mix (NEB #E5315) was used to amplify the generated cDNA.

### Raptamer FISH

The steps after immobilization were performed as described previously in the Shaffer, S. M.; Wu, M. T.; Levesque, M. J.; Raj, A. Turbo FISH: A Method for Rapid Single Molecule RNA FISH. PLoS One 2013, 8 (9), e75120 (the contents of which are herein incorporated by reference in their entirety). The coverglass was washed 3 times with the binding buffer after virions capture. To fix virus samples, −20° C. methanol was added to samples, which were then incubated at −20° C. for 10 min. 500 μM of probe mixture was mixed with the hybridization buffer (10% dextran sulfate/10% formamide/2×SSC) and incubated with the fixed samples for 5 min. The samples were washed 2 times with the wash buffer (2×SSC, 10% formamide) then 2×SSC was added for subsequent imaging.

### Image Acquisition

Microscopy was performed using a Nikon inverted research microscope eclipse Ti2-E/Ti2-E/B using a Plan Apo λ 20×/0.75 objective or Plan Apo λ 100×/1.45 oil objective. The Epi-fi LED illuminator linked to the microscope assured illumination and controlled the respective brightness of four types of LEDs of different wavelengths. Images were acquired using the Neutral Density (ND16) filter for probes coupled with Alexa 488, Alexa 594, Alexa 647, cy3. Images were acquired and processed using ImageJ. Images acquired using the Neutral Density (ND16) filter are false-colored gray. Nucleus of live cells were stained by NucBlue™ Live Cell Stain ReadyPorbes™ reagent (ThermoFisher #R38605).

### Image Analysis and Quantification

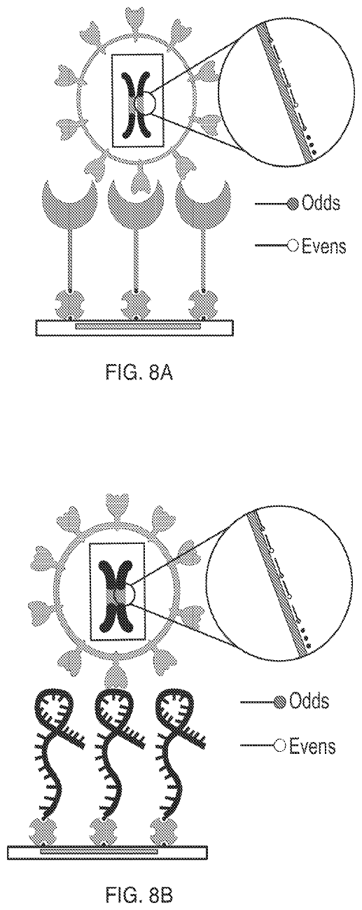

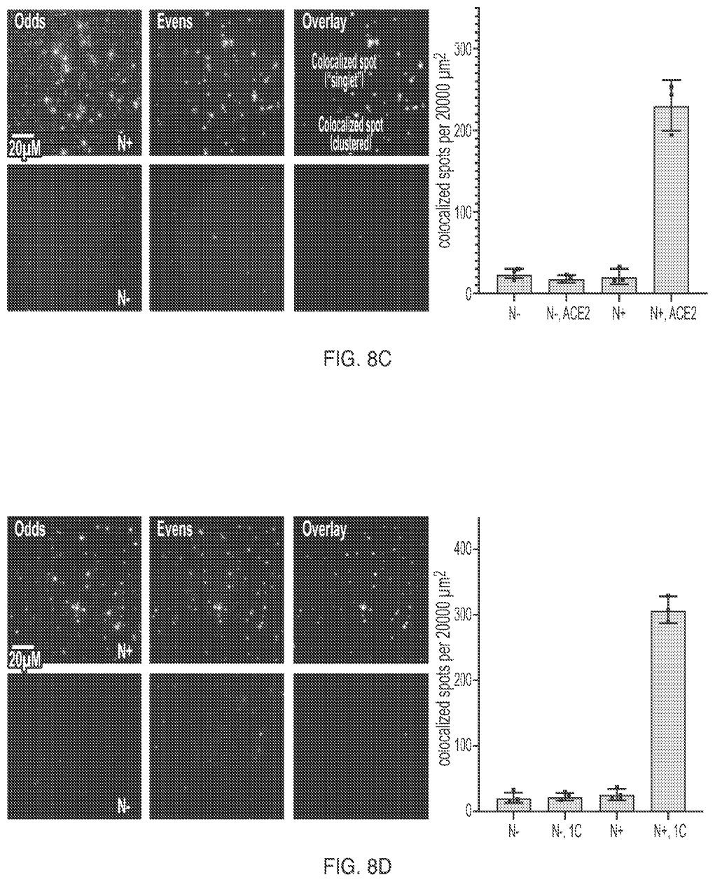

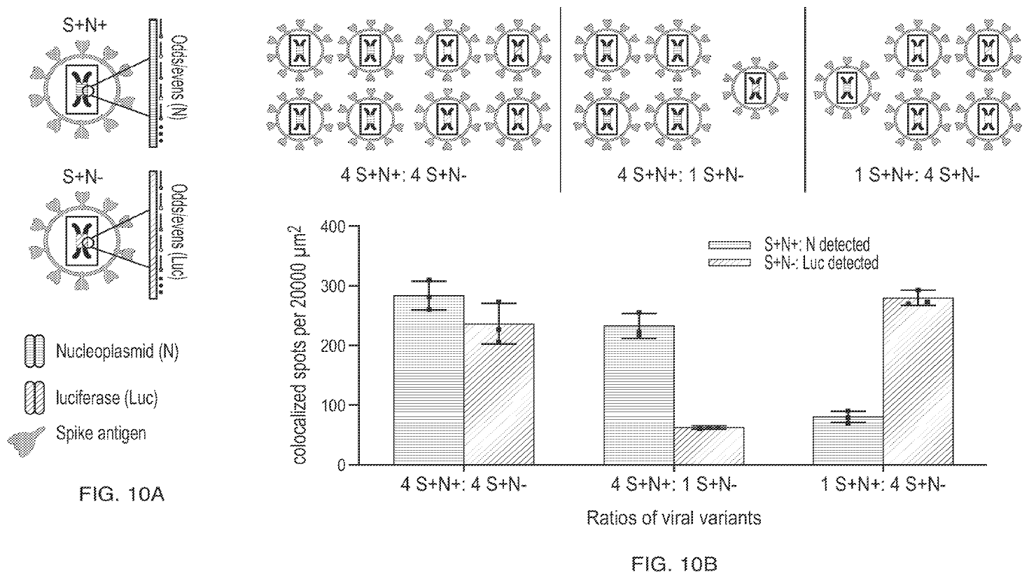

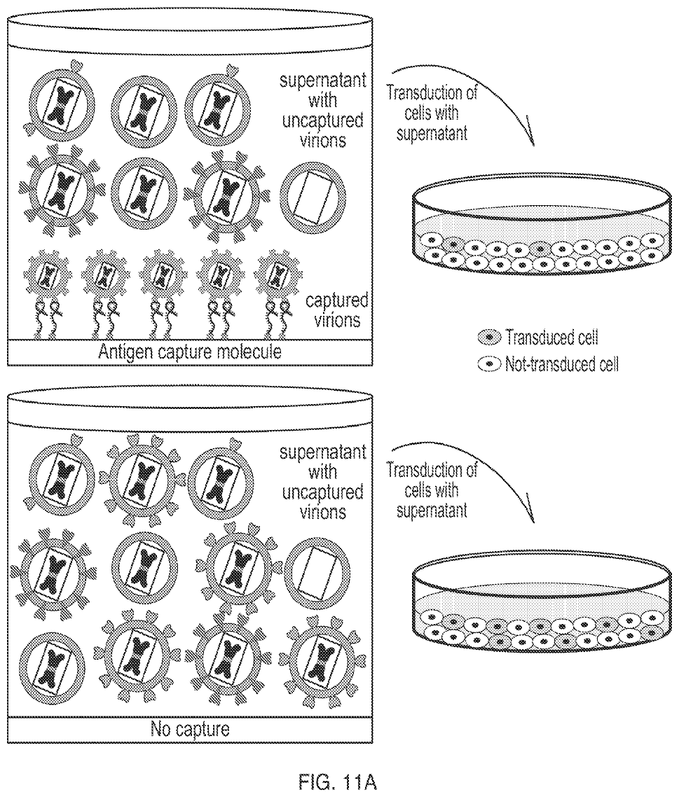

After imaging, the data were put through an image analysis pipeline for semi-automated spot recognition. The pipeline, developed in Matlab, can be divided into three main steps of (i) binarization, (ii) colocalization and (iii) size thresholding. Briefly, in the first binarization step we manually determine a threshold for each fluorescence channel so the number of spots can be localized well in positive control. Images of each channel share the same thresholding level, which is applied to the images to binarize them. The binarized images are then overlapped and intersected in order to find the common spots to the two channels (FIGS. 8A-8D and FIGS. 10A-10B). Eventually, in the last step a cut-off level for the particle area was set, in order to exclude viral aggregates from the analysis and limit the colocalized group to just individual virions (FIG. 12).

### Two-Step R T-qPCR

Use SuperScript III Reverse Transcriptase (FisherSci #18-080-044), RNaseOUT (FischerSci #10777-019) and Luna qPCR (NEB #M3004). Reverse transcription was performed by mixing 7.5 μl DNase digestion product (10 pg-500 ng mRNA), 1 μl 10 mM dNTPs, 1 μl 2 μM CMV-reverse primer, and Nuclease free water up to 13 μl, followed by incubation at 65° C. for 3 min and ice for 1 min. The sample was placed on ice and a mix containing 1 μl 0.1M DTT, 4 μl 5× First-Strand Buffer, 1 μl RNaseOUT, 1 μl SuperScript III RT (200 U/ul) (replace with Nuclease free water for RT negative control) and water up to 7 μl was added. The samples were then incubated at 50° C. for 60 min followed by 70° C. for 15 min. Amplification of 1 μl cDNA (RT mix) using primers and probes described in Table 1 was performed using in a 20 μl reaction containing 10 μl Master Mix, 0.8 μl forward, 0.8 μl reverse 10 μM CMV primers, 0.4 μl 10 μM CMV probe and water up to 20 μl. The thermal cycling steps were: 95° C. for 1 min, and 45 cycles of 95° C. for 15 s and 60° C. for 30 s. qPCR was performed on a CFX96™ Optics Module,
C1000 Touch™ Thermal Cycler (BIO-RAD) using the CFX Manager™ Software v3.1.

### One-Step R T-PCR

For reverse transcription and PCR the OneTaq RT-PCR mix (NEB #E5315) was used according to the manufacturer's instructions. Reactions of 25 μl were formed by mixing DNA digestion product (mRNA up to 1 μg), OneTaq One-Step Reaction Mix (2×) 12.5 μl, OneTaq One-Step Enzyme Mix (25×) 1 μl, 1 μl 10 μM forward primer and reverse primer listed in Table 2 and Nuclease free water up to 25 μl. The thermal cycling steps were: 48° C. for 2 min, 94° C. for 1 min and 30 cycles of 94° C. for 15 s and 50° C. for 30 s, 68° C. for 75 s. After cycles final extension was 68° C. for 5 min and hold at 10° C. RT-PCR was performed on a T100 Thermal Cycler machine (BIO-RAD).

### Sequences

TABLE 1
Primers and Probes used for RT-qPCR
to quantify captured virions

| | Amplicon | | |
|---|---|---|---|
| | length | | |
| Name | (bp) | Description | Sequence 5′ to 3′ |
| CMV | 104 bp | Forward | TCACGGGGATTTCCAAGT |
| promoter | | | CTC |
| | | | (SEQ ID NO: 1) |
| | | Reverse | AATGGGGCGGAGTTGTTA |
| | | | CGAC |
| | | | (SEQ ID NO: 2) |
| | | Probe | /56-FAM/AAACAAACT/ |
| | | | ZEN/CCCATTGACGTCA/ |
| | | | 3IaBkFQ/ |
| | | | (SEQ ID NO: 3) |

TABLE 2
Primers used for RT-PCR to amplify N gene

| | Amplicon | | |
|---|---|---|---|
| | length | | |
| Name | (bp) | Description | Sequence 5′ to 3′ |
| Nucleo- | 1260 bp | Forward | ATGTCTGATAATGGACCCCA |
| plasmids | | | (SEQ ID NO: 4) |
| | | Reverse | TTAGGCCTGAGTTGAGTC |
| | | | (SEQ ID NO: 5) |

Example plasmids used to generate lentivirus particles are listed below and in the incorporated sequence listing. (SEQ ID NOS: 6-14). All the modified sequences were sequence-verified. See also description in Liu, Y. et al. Paired aptamer capture and FISH detection of individual virions enables cell-free determination of infectious titer. bioRxiv 2022.11.13.516306 (2022) doi: 10.1101/2022.11.13.516306, along with the Supporting Information, Supplementary methods and Supplementary Note: Sequences, all incorporated herein by reference in entirety.

Vector Hgpm2 is SEQ ID NO: 6.

Vector Tat1b is SEQ ID NO: 7.

Vector Rev1b is SEQ ID NO: 8.

Vector Luc2-ZsGreen, variant sequence including CMV promoter, Luciferase and ZsGreen is SEQ ID NO: 9.

Vector Spike G614419. Plasmid to increase spike protein infectivity was prepared by Gibson assembly is SEQ ID NO: 10. Modified sequence from wild type Spike is SEQ ID NO: 11.

Vector CoV2Ngene-Luc2-ZsGreen. Plasmids to insert N gene of SARS-CoV-2 were prepared by Gibson assembly is SEQ ID NO: 12. Variant sequences include the N gene and ZsGreen sequence. Inserted N gene sequence is SEQ ID NO: 13.

Vector VSV-G is SEQ ID NO: 14.

### EXEMPLIFICATION

### Example 1. Capture and Sandwich ELISA Detection of Recombinant Spike Protein with Anti-Spike Aptamer

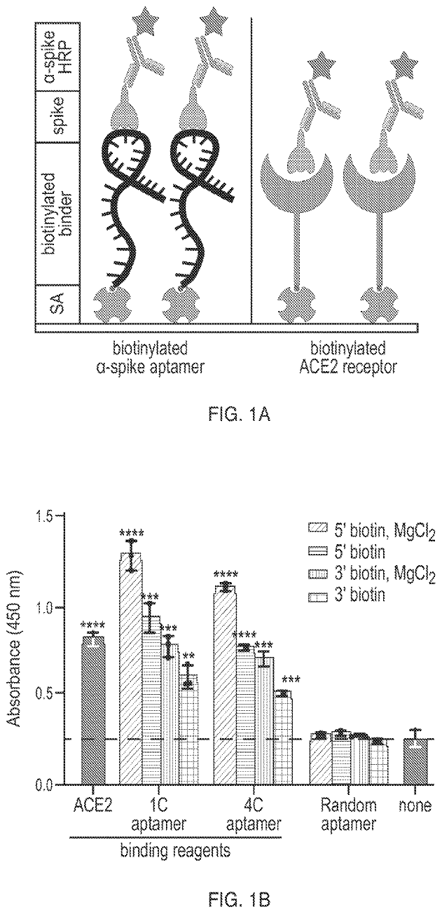

DNA aptamer sequences (1 C and 4 C) that were computationally predicted to bind to the receptor binding domain of the SARS-CoV-2 Spike protein were selected. The K_{d} values for these aptamers towards the spike trimer were reported as 5.8 nM and 19.9 nM respectively (Song, Y.; Song, J.; Wei, X.; Huang, M.; Sun, M.; Zhu, L.; Lin, B.; Shen, H.; Zhu, Z.; Yang, C. Discovery of Aptamers Targeting Receptor-Binding Domain of the SARS-CoV-2 Spike Glycoprotein; the contents of which are herein incorporated by reference in their entirety). To evaluate the binding properties of the aptamers for the assay, aptamers biotinylated on either the 5′ and 3′ ends were immobilized to streptavidin-coated polystyrene plates (FIG. 1A, left). As a positive control, biotinylated ACE2 protein was immobilized to the streptavidin-coated polystyrene plates. As a negative control, random aptamer sequences were used (FIG. 1A, right). Recombinant SARS-CoV-2 Spike protein was applied to the immobilized capture reagents and a sandwich ELISA was performed using an HRP-conjugated anti-Spike antibody followed by colorimetric detection.

The 1 C aptamers were observed to have a stronger affinity towards the spike protein than the 4 C aptamers. 5′ biotin-modified 1 C had a 4.724±0.079-fold increase of binding and 5′ biotin-modified 4 C aptamer had a 4.077±0.083-fold increase of binding as random aptamer in the presence of MgCl₂·5′ biotin-modified aptamers captured 1.615±0.055-fold more spike protein than the 3′ modified aptamers. The presence of MgCl₂ also had a positive effect on the quantity of spike protein captured with a 1.373±0.029 fold increase in absorbance over the condition where MgCl₂ was omitted, consistent with previous work (Song, Y.; Song, J.; Wei, X.; Huang, M.; Sun, M.; Zhu, L.; Lin, B.; Shen, H.; Zhu, Z.; Yang, C. Discovery of Aptamers Targeting Receptor-Binding Domain of the SARS-CoV-2 Spike Glycoprotein; Jahan, M.; Uline, M. J. Quantifying Mg2+ Binding to SsDNA Oligomers: A Self-Consistent Field Theory Study at Varying Ionic Strengths and Grafting Densities. Polymers 2018, 10 (12); Carothers, J. M.; Goler, J. A.; Kapoor, Y.; Lara, L.; Keasling, J. D. Selecting RNA Aptamers for Synthetic Biology: Investigating Magnesium Dependence and Predicting Binding Affinity. Nucleic Acids Res. 2010, 38 (8), 2736-2747; the contents of which are herein incorporated by reference in their entirety) (FIG. 1B). Overall, 5′ biotin-modified 1 C aptamers exhibit the highest affinity towards recombinant spike protein with MgCl₂. This condition was used for the following experiments.

### Example 2. Capture and Detection of Spike-Pseudotyped Lentiviruses with Anti-Spike Aptamer Using RT-qPCR

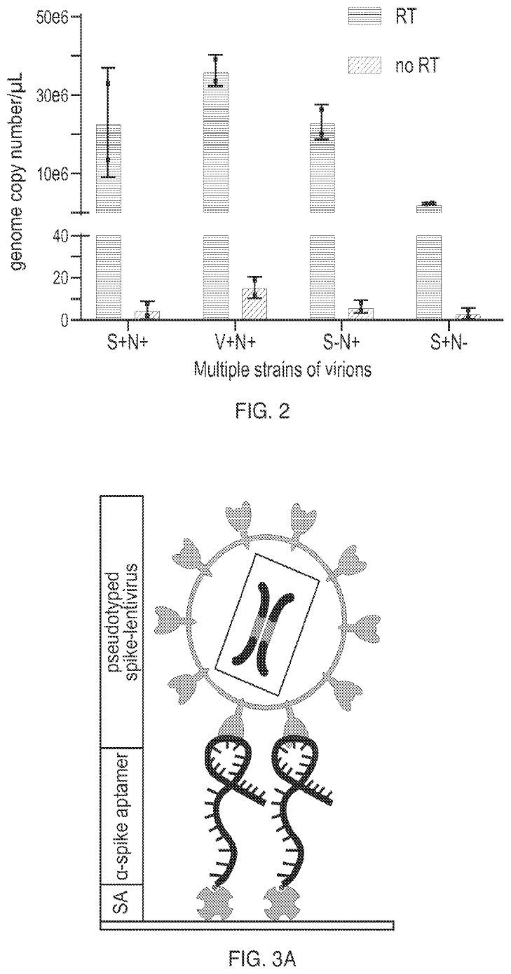

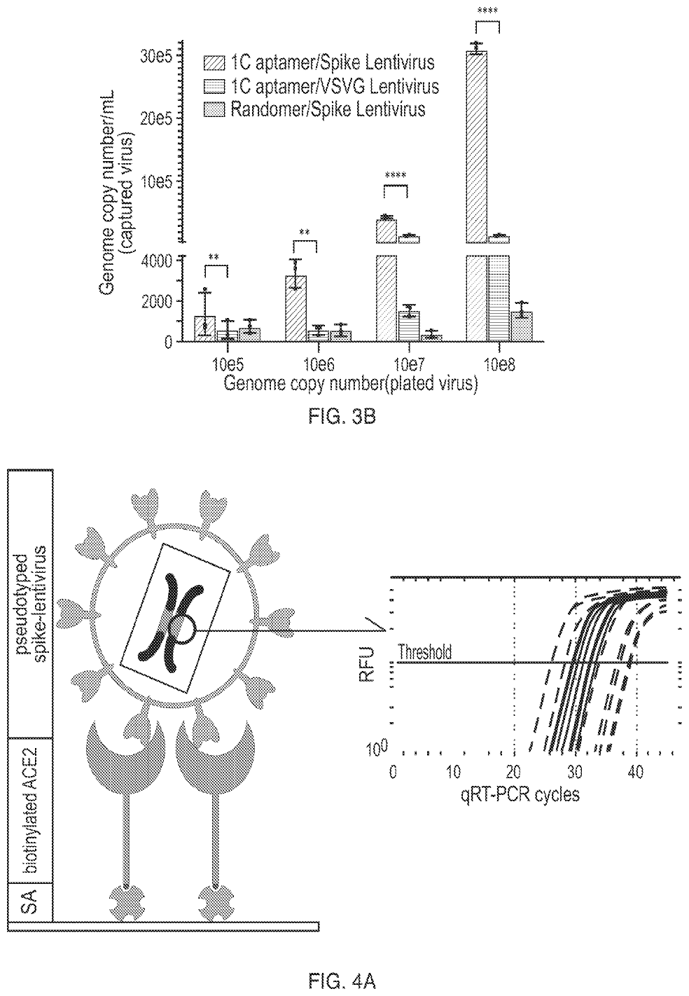

To assess the binding efficiency of virions to the immobilized aptamers, SARS-CoV-2 spike-pseudotyped lentiviral particles were produced according to the methods of Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5) (the contents of which are herein incorporated by reference in their entirety). ACE2 and anti-spike aptamer 1 C were immobilized on streptavidin-coated polystyrene plates and exposed to lentivirus virions to assess the capture efficiency. A random aptamer-coated plate and lentiviral particles pseudotyped with Vesicular Stomatitis Virus Glycoprotein (VSVG) were used as negative controls to assess the nonspecific binding of the virus to ACE2 and 1 C aptamer. The spike-pseudotyped lentivirus and the VSVG-pseudotyped lentivirus were added to the plates at concentrations ranging from 10⁵ to 10⁸ genome copies (determined by RT-qPCR; FIG. 2). After washing, the RNA from captured virions was extracted and RT-qPCR targeting the CMV promoter was performed (common between both lentivirus vectors) to determine capture efficiency (FIG. 3A).

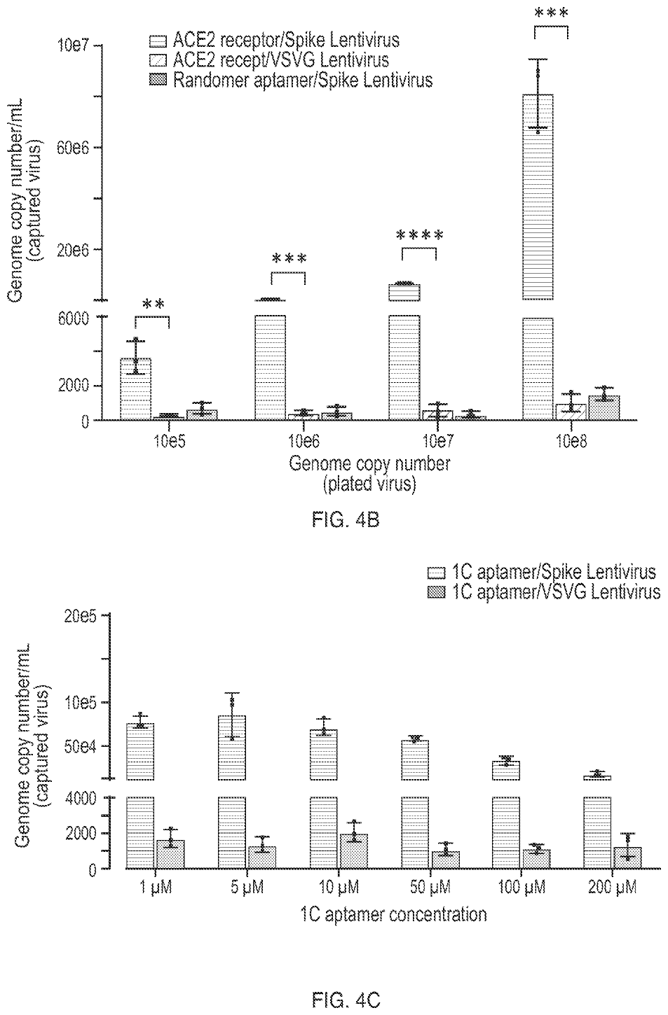

Specific capture of spike-pseudotyped lentivirus was detected with the 1 C capture reagent at a minimum concentration of 10³ copies per μl, showing a 2.412-fold increase in genome-containing units over the nonspecific virus captured by the random aptamer. When the concentration was increased to 10⁶ genome-containing units per μl, the capture efficiency increased, showing a 208.292-fold increase in genome copies over the nonspecific virus captured by the random aptamer (FIG. 3B). The same capture efficiency was also observed in the ACE2 protein (FIG. 4A, 4B).

A range of aptamer concentrations were used to coat the streptavidin plate to determine the optimal concentration for virion capture. The capture, RNA extraction, reverse transcription, and qPCR were performed, and six aptamer concentrations ranging from 1 μM-200 μM were selected. A 45.688-fold higher binding for the 5 μM aptamer concentration was observed over the VSV-G virus and a 10.064-fold higher binding to 200 μM aptamer concentration. Aptamer binding became saturated at around 10 μM (this concentration was used for all following experiments) and a 40-fold increase in spike pseudotyped virus captured over VSV-G virus at 10 μM was observed (FIG. 4C).

### Example 3. Raptamer FISH Detection of Spike-Pseudotyped Lentiviruses with 1 C Aptamer

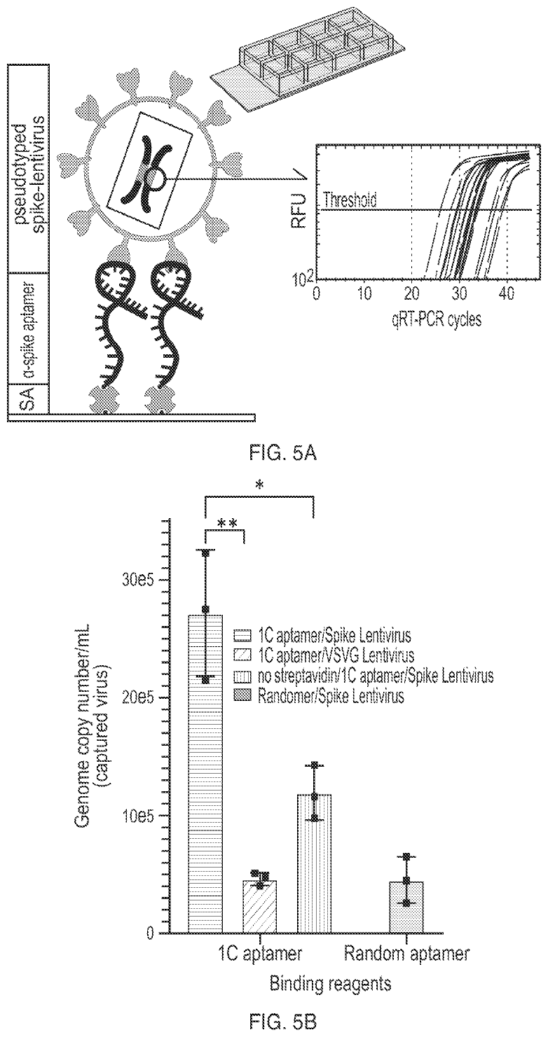

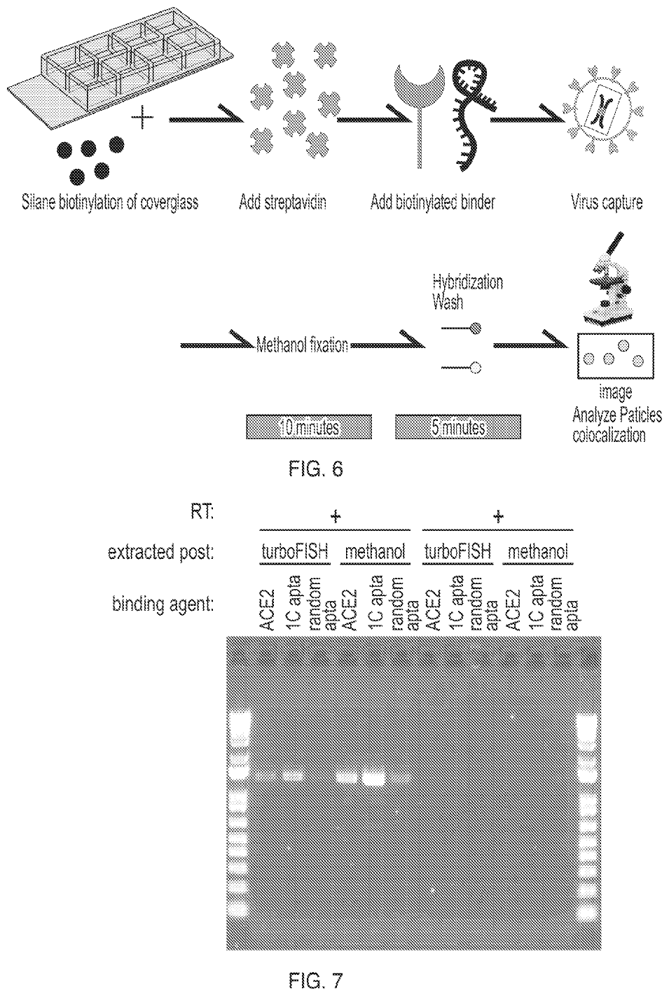

After validating that the disclosed system can capture viruses on polystyrene plates and chambered coverglass (FIG. 5A, 5B), the utility of fluorescence in situ hybridization (FISH) to detect individual virions that are captured by aptamers in a method dubbed rapid-aptamer FISH was tested (raptamer FISH; FIG. 6). Previous work (Chou, Y. Y.; Vafabakhsh, R.; Doğanay, S.; Gao, Q.; Ha, T.; Palese, P. One Influenza Virus Particle Packages Eight Unique Viral RNAs as Shown by FISH Analysis. Proc. Natl. Acad. Sci. U.S.A. 2012, 109 (23), 9101-9106; Chou, Y. Y.; Lionnet, T. Single-Molecule Sensitivity RNA FISH Analysis of Influenza Virus Genome Trafficking. Methods Mol. Biol. 2018, 1836, 195-211; the contents of which are herein incorporated by reference in their entirety) of detecting virions relies on single molecule FISH (smFISH), which employs hybridization times of up to 16 h; however, an alternative method called TurboFISH (described in Shaffer, S. M.; Wu, M. T.; Levesque, M. J.; Raj, A. Turbo FISH: A Method for Rapid Single Molecule RNA FISH. PLoS One 2013, 8 (9), e75120; the contents of which are herein incorporated by reference in their entirety) can decrease the hybridization time to 5 minutes to achieve fast detection.

Confirmation that the virions would retain their integrity after fixation and permeabilization by capturing the virus and then performing RT-PCR on the captured virions post-methanol fixation and post-TurboFISH was determined (FIG. 7). The chambered coverglass was biotinylated by silane-biotin, and virions were captured. Briefly, the virus was fixed with methanol after specific capture, and smFISH probes targeting the SARS-CoV-2 nucleocapsid gene (N gene) were introduced and hybridized. After hybridization, there were further wash steps followed by epifluorescent microscopy (FIG. 6, 8A, 8B).

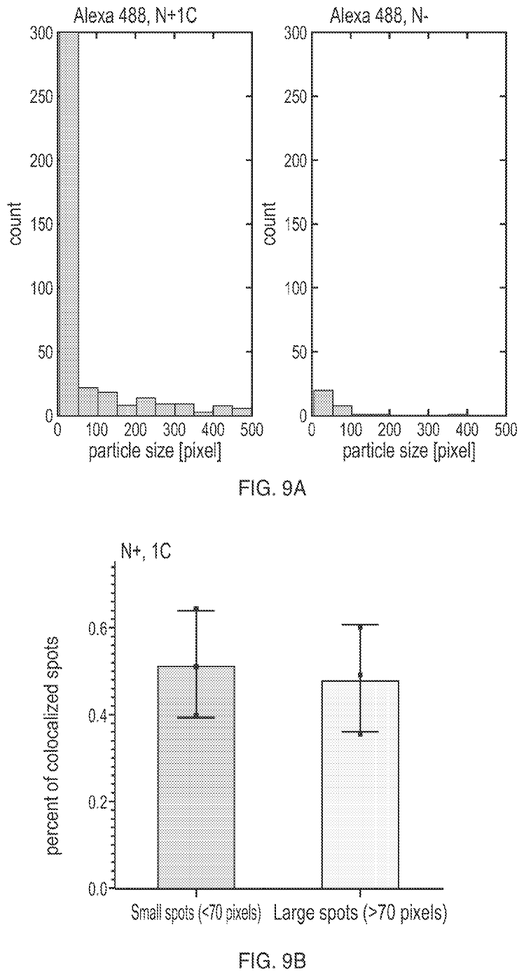

The size of the raptamer FISH spots was determined to be 1-500 pixels by particle analysis in Matlab. Lentivirus is known to aggregate. Approximately 79.572% of detected spots were 1-70 pixels in diameter, showing up only in the samples where virus was added. Therefore, it was reasoned that colocalized spots in the 1-70-pixel range were likely individual virions. It was observed that 20.428% of spots were beyond 70 pixels and these large spots were probable viral aggregates (FIG. 9A, 9B). The large particles (spots more than 70 pixels) showed the same colocalization rates as the small particles, indicating that the viral aggregates were infectious (FIG. 9A, 9B).

To confirm the detection of a true viral particle, two sets of probes (odds and evens) were used to mitigate the detection of false positive spots. The specificity of probes for N gene was demonstrated previously in Acheampong, K. K.; Schaff, D. L.; Emert, B. L.; Lake, J.; Reffsin, S.; Shea, E. K.; Comar, C. E.; Litzky, L. A.; Khurram, N. A.; Linn, R. L.; Feldman, M.; Weiss, S. R.; Montone, K. T.; Cherry, S.; Shaffer, S. M. Subcellular Detection of SARS-CoV-2 RNA in Human Tissue Reveals Distinct Localization in Alveolar Type 2 Pneumocytes and Alveolar Macrophages. MBio 2022, e0375121 (the contents of which herein are incorporated by reference in their entirety). To confirm specific viral detection, raptamer FISH was applied to a series of negative controls, including the slides not treated with ACE2 protein and the slides not treated with 1 C aptamers. Additionally, viruses whose genome contains the N gene (N+) or not (N−) were tested (FIG. 8C, 8D).

For the functionalized slides treated with ACE2 or 1 C aptamer and in the presence of N+ virus, we observed a 10.755-fold and 12.780-fold increase of colocalized spots, respectively, as compared to the samples without binding agents and N gene (FIG. 8C, 8D).

### Example 4. Simultaneous Raptamer FISH Detection of Multiple Virus Strains

To test whether the raptamer FISH system can detect multiple strains, the assay was performed on a mixed population of spike coated virions containing two distinct genomes. N+ virions, as described herein, were mixed in different ratios with virions containing the luciferase gene (N−). The N gene probe targets the N gene of spike-coated lentivirus (S+N+), and the luciferase probe targets the luciferase of spike-coated lentivirus without the N gene (S+N−) was combined to detect two types of virions simultaneously. Two sets of probes (odds and evens) with two dyes for each target were used to detect the colocalized FISH spots (FIG. 10A). Virions were mixed with different concentration ratios, then captured, fixed, and detected by TurboFISH simultaneously (FIG. 10B).

Three concentration ratios based on RT-qPCR of the two virion types were used to test our system: 4:4, 1:4, and 4:1 of S+N+: S+N−. For the 4:4 ratio, the ratio of colocalized spots of S+N+ to S+N− was 1.209:1. For the 4:1 ratio, the ratio of colocalized spots of S+N+ to S+N− was 3.679:1. For the 1:4 ratio, the ratio of colocalized spots of S+N+ to S+N− was 1:3.498, consistent with the concentration ratio of the two virions we added, (FIG. 10B) indicating that raptamer FISH can simultaneously detect multiple virion types.

### Example 5. Functional Titering of Remaining Supernatant from Virions Captured by Aptamers and ACE2 Receptors

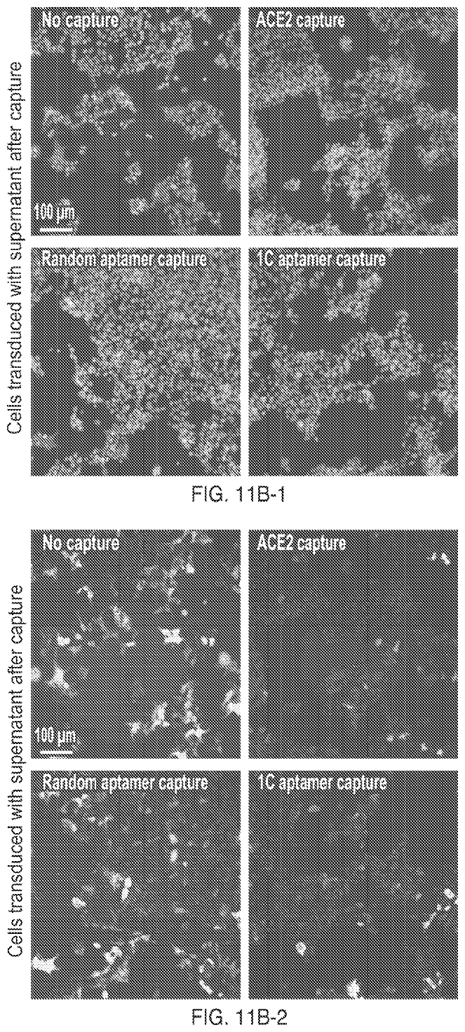

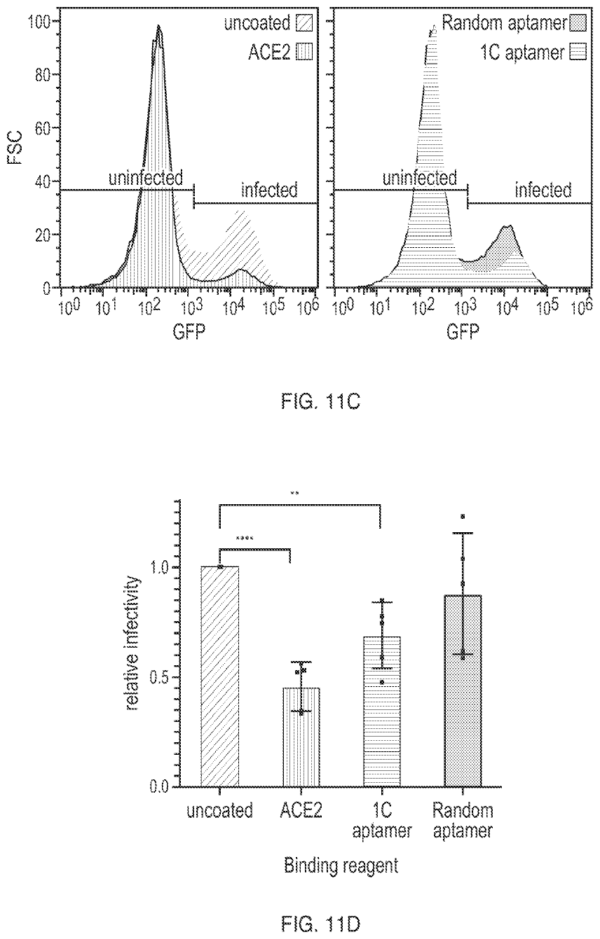

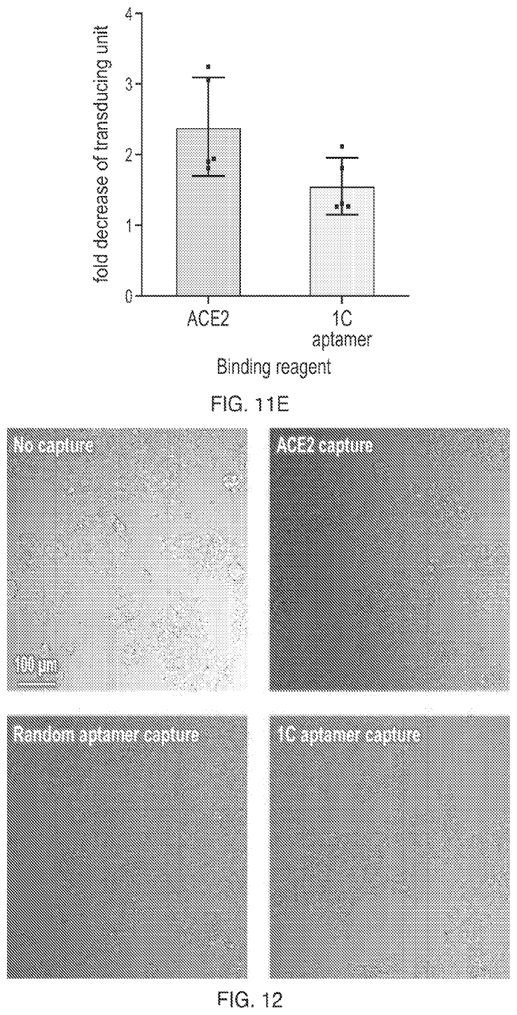

Infectious titering relies on cell-based assays. Still accuracy and consistency of such methods heavily depend on cell condition. The system disclosed herein achieves a cell-free proxy for infectious titers. To test whether the infectious viral particles are, indeed, getting captured, the infectivity of the supernatant after incubating the virus on the ACE2 receptor and the 1 C aptamer was assessed. The virion capture steps of aptamers and ACE2 were performed as described in FIG. 3A and FIG. 3B. The uncaptured virions in the supernatant were aspirated and used to transduce ACE2-expressing HEK293T cells (HEK293T-ACE2). After incubating HEK293T-ACE2 cells with the virus for two days, the percentage of infected cells was measured with flow cytometry via ZsGreen expression from the viral genome (FIG. 11A). Wildtype HEK293FT cells were infected simultaneously to set up the gate of uninfected cells. Plates not coated with aptamers (uncoated) were used to do normalization and measure the total amount of infectious particles. The percentage of infected cells was used to quantify the infectivity of different samples. Random aptamers were used as a negative control.

The percentage of infected cells decreased significantly after virion capture by ACE2 and 1 C aptamers, compared to the infectivity of viruses that were not captured, suggesting that the infectious particles are being captured. Random aptamers had similar infectivity as the no capture group, which indicated that such capture is highly specific (FIG. 11B-1, 11B-2, FIG. 12). Based on the flow cytometry data, two populations of uninfected/infected cells were gated in the histogram (FIG. 11C). There was a 65.751% decrease in the percent of infected cells in the ACE2 group compared to the uncoated group (FIG. 11C) and a 36.863% decrease in the percent of infected cells in the 1 C aptamers group compared to the random aptamers group (FIG. 11C). The random aptamer capture was comparable to the non-specific capture on uncoated plates. The percentage of infected cells in ACE2, 1C, and random aptamers were normalized to that of the uncoated group (FIG. 11D). Percentages of infected cells were transformed to TU/mL using the viral quantification formula. A 2.392-fold reduction and a 1.552-fold reduction in TU/mL were observed in the ACE2 group and 1 C aptamer group, respectively, compared to the uncoated group (FIG. 11E).

### Example 6. Relationship Between Raptamer FISH Detection of Infectious Virions and Functional Viral Titer

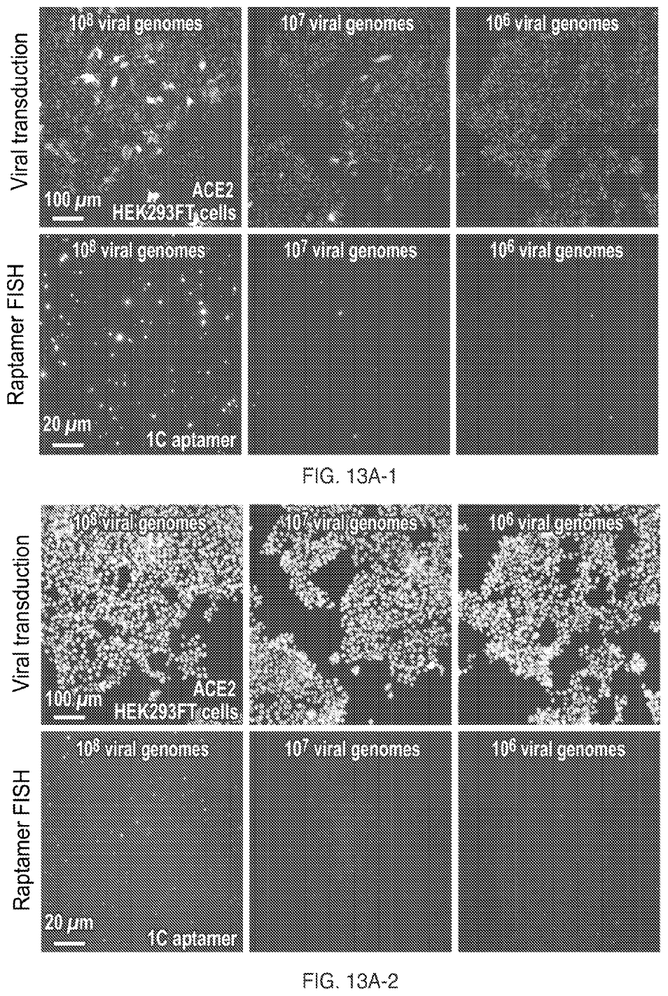

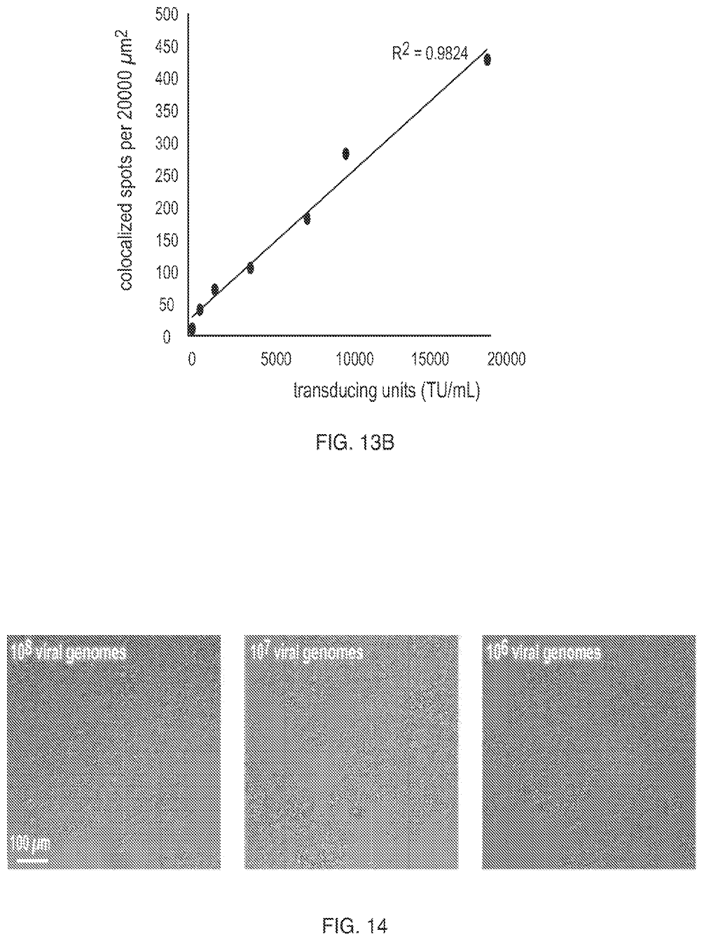

To assess the quantitative accuracy of the presently disclosed cell-free viral detection system, the functional virus titer and the corresponding raptamer FISH quantification were measured. HEK293T-ACE2 cells were transduced with spike-pseudotyped lentivirus with N gene at seven concentrations ranging from 106 to 108 genome units (determined by qRT-PCR; FIG. 13A-1, FIG. 13A-2). The percentage of infected cells (zsgreen positive cell) was transformed to TU/mL as done by FIG. 11. The same virus concentration (genome units) was applied to glass slides with immobilized 1 C aptamer to capture the spike-pseudotyped lentivirus with the N gene, followed by detection of the N gene with raptamer FISH (FIG. 13A-1, FIG. 13A-2). The colocalized spots per 2000 μm² increased proportionally with the transducing units (TU/mL) for the same genome containing units (R²=0.9824; FIG. 13B, FIG. 14).

### REFERENCES

(1) de la Rica, R.; Stevens, M. M. Plasmonic ELISA for the Ultrasensitive Detection of Disease Biomarkers with the Naked Eye. Nat. Nanotechnol. 2012, 7 (12), 821-824.
(2) Souf, S. Recent Advances in Diagnostic Testing for Viral Infections. Bioscience Horizons: The International Journal of 2016.
(3) Corman, V. M.; Landt, O.; Kaiser, M.; Molenkamp, R.; Meijer, A.; Chu, D. K. W.; Bleicker, T.; Brünink, S.; Schneider, J.; Schmidt, M. L.; Mulders, D. G.; Haagmans, B. L.; van der Veer, B.; van den Brink, S.; Wijsman, L.; Goderski, G.; Romette, J. L.; Ellis, J.; Zambon, M.; Peiris, M.; Goossens, H.; Reusken, C.; Koopmans, M. P. G.; Drosten, C. Detection of 2019 Novel Coronavirus (2019-NCOV) by Real-Time RT-PCR. Eurosurveillance. 2020. https://doi.org/10.2807/1560-7917.es.2020.25.3.2000045.
(4) Huang, H. S.; Tsai, C. L.; Chang, J.; Hsu, T. C.; Lin, S.; Lee, C. C. Multiplex PCR System for the Rapid Diagnosis of Respiratory Virus Infection: Systematic Review and Meta-Analysis. Clin. Microbiol. Infect. 2018, 24 (10), 1055-1063.
(5) Klasse, P. J. Molecular Determinants of the Ratio of Inert to Infectious Virus Particles. Prog. Mol. Biol. Transl. Sci. 2015, 129, 285-326.
(6) Carpenter, J. E.; Henderson, E. P.; Grose, C. Enumeration of an Extremely High Particle-to-PFU Ratio for Varicella-Zoster Virus. J. Virol. 2009, 83 (13), 6917-6921.
(7) Uchino, J.; Curiel, D. T.; Ugai, H. Species D Human Adenovirus Type 9 Exhibits Better Virus-Spread Ability for Antitumor Efficacy among Alternative Serotypes. PLoS One 2014, 9 (2), e87342.
(8) Layne, S. P.; Merges, M. J.; Dembo, M.; Spouge, J. L.; Conley, S. R.; Moore, J. P.; Raina, J. L.; Renz, H.; Gelderblom, H. R.; Nara, P. L. Factors Underlying Spontaneous Inactivation and Susceptibility to Neutralization of Human Immunodeficiency Virus. Virology 1992, 189 (2), 695-714.
(9) Joynt, G. M.; Wu, W. K. Understanding COVID-19: What Does Viral RNA Load Really Mean? Lancet Infect. Dis. 2020, 20 (6), 635-636.
(10) Mendoza, E. J.; Manguiat, K.; Wood, H.; Drebot, M. Two Detailed Plaque Assay Protocols for the Quantification of Infectious SARS-CoV-2. Curr. Protoc. Microbiol. 2020, 57 (1), ecpmc105.
(11) Amarilla, A. A.; Modhiran, N.; Setoh, Y. X.; Peng, N. Y. G.; Sng, J. D. J.; Liang, B.; McMillan, C. L. D.; Freney, M. E.; Cheung, S. T. M.; Chappell, K. J.; Khromykh, A. A.; Young, P. R.; Watterson, D. An Optimized High-Throughput Immuno-Plaque Assay for SARS-CoV-2. Front. Microbiol. 2021, 12, 625136.
(12) LaBarre, D. D.; Lowy, R. J. Improvements in Methods for Calculating Virus Titer Estimates from TCID50 and Plaque Assays. J. Virol. Methods 2001, 96 (2), 107-126.
(13) Geraerts, M.; Willems, S.; Baekelandt, V.; Debyser, Z.; Gijsbers, R. Comparison of Lentiviral Vector Titration Methods. BMC Biotechnol. 2006, 6, 34.
(14) Raj, A.; van den Bogaard, P.; Rifkin, S. A.; van Oudenaarden, A.; Tyagi, S. Imaging Individual MRNA Molecules Using Multiple Singly Labeled Probes. Nat. Methods 2008, 5 (10), 877-879.
(15) Shaffer, S. M.; Joshi, R. P.; Chambers, B. S.; Sterken, D.; Biaesch, A. G.; Gabrieli, D. J.; Li, Y.; Feemster, K. A.; Hensley, S. E.; Issadore, D.; Raj, A. Multiplexed Detection of Viral Infections Using Rapid in Situ RNA Analysis on a Chip. Lab Chip 2015, 15 (15), 3170-3182.
(16) Shaffer, S. M.; Wu, M. T.; Levesque, M. J.; Raj, A. Turbo FISH: A Method for Rapid Single Molecule RNA FISH. PLoS One 2013, 8 (9), e75120.
(17) Wichgers Schreur, P. J.; Kortekaas, J. Single-Molecule FISH Reveals Non-Selective Packaging of Rift Valley Fever Virus Genome Segments. PLoS Pathog. 2016, 12 (8), e1005800.
(18) Acheampong, K. K.; Schaff, D. L.; Emert, B. L.; Lake, J.; Reffsin, S.; Shea, E. K.; Comar, C. E.; Litzky, L. A.; Khurram, N. A.; Linn, R. L.; Feldman, M.; Weiss, S. R.; Montone, K. T.; Cherry, S.; Shaffer, S. M. Subcellular Detection of SARS-CoV-2 RNA in Human Tissue Reveals Distinct Localization in Alveolar Type 2 Pneumocytes and Alveolar Macrophages. MBio 2022, e0375121.
(19) Chou, Y. Y.; Vafabakhsh, R.; Doğanay, S.; Gao, Q.; Ha, T.; Palese, P. One Influenza Virus Particle Packages Eight Unique Viral RNAs as Shown by FISH Analysis. Proc. Natl. Acad. Sci. U.S.A 2012, 109 (23), 9101-9106.
(20) Hepp, C.; Shiaelis, N.; Robb, N. C.; Vaughan, A.; Matthews, P. C.; Stoesser, N.; Crook, D.; Kapanidis, A. N. Viral Detection and Identification in 20 Min by Rapid Single-Particle Fluorescence in-Situ Hybridization of Viral RNA. Sci. Rep. 2021, 11 (1), 19579.
(21) Yeh, Y. T.; Gulino, K.; Zhang, Y.; Sabestien, A.; Chou, T. W.; Zhou, B.; Lin, Z.; Albert, I.; Lu, H.; Swaminathan, V.; Ghedin, E.; Terrones, M. A Rapid and Label-Free Platform for Virus Capture and Identification from Clinical Samples. Proc. Natl. Acad. Sci. U.S.A 2020, 117 (2), 895-901.
(22) Song, Y.; Song, J.; Wei, X.; Huang, M.; Sun, M.; Zhu, L.; Lin, B.; Shen, H.; Zhu, Z.; Yang, C. Discovery of Aptamers Targeting Receptor-Binding Domain of the SARS-CoV-2 Spike Glycoprotein. https://doi.org/10.26434/chemrxiv.12053535.
(23) Jahan, M.; Uline, M. J. Quantifying Mg2+ Binding to SsDNA Oligomers: A Self-Consistent Field Theory Study at Varying Ionic Strengths and Grafting Densities. Polymers 2018, 10 (12). https://doi.org/10.3390/polym10121403.
(24) Carothers, J. M.; Goler, J. A.; Kapoor, Y.; Lara, L.; Keasling, J. D. Selecting RNA Aptamers for Synthetic Biology: Investigating Magnesium Dependence and Predicting Binding Affinity. Nucleic Acids Res. 2010, 38 (8), 2736-2747.
(25) Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12 (5). https://doi.org/10.3390/v12050513.
(26) Ci, Y.; Yang, Y.; Xu, C.; Shi, L. Vesicular Stomatitis Virus G Protein Transmembrane Region Is Crucial for the Hemi-Fusion to Full Fusion Transition. Sci. Rep. 2018, 8 (1), 10669.
(27) Chou, Y. Y.; Lionnet, T. Single-Molecule Sensitivity RNA FISH Analysis of Influenza Virus Genome Trafficking. Methods Mol. Biol. 2018, 1836, 195-211.
(28) Perry, C.; Rayat, A. C. M. E. Lentiviral Vector Bioprocessing. Viruses 2021, 13 (2). https://doi.org/10.3390/v13020268.
(29) Arnaout, R.; Lee, R. A.; Lee, G. R.; Callahan, C.; Yen, C. F.; Smith, K. P.; Arora, R.; Kirby, J. E. SARS-CoV2 Testing: The Limit of Detection Matters. bioRxiv 2020. https://doi.org/10.1101/2020.06.02.131144.
(30) Arnaout, R.; Lee, R. A.; Lee, G. R.; Callahan, C.; Cheng, A.; Yen, C. F.; Smith, K. P.; Arora, R.; Kirby, J. E. The Limit of Detection Matters: The Case for Benchmarking Severe Acute Respiratory Syndrome Coronavirus 2 Testing. Clin. Infect. Dis. 2021, 73 (9), e3042-e3046.
(31) Harvey, W. T.; Carabelli, A. M.; Jackson, B.; Gupta, R. K.; Thomson, E. C.; Harrison, E. M.; Ludden, C.; Reeve, R.; Rambaut, A.; COVID-19 Genomics UK (COG-UK) Consortium; Peacock, S. J.; Robertson, D. L. SARS-CoV-2 Variants, Spike Mutations and Immune Escape. Nat. Rev. Microbiol. 2021, 19 (7), 409-424.
(32) Ning, X.; Luckenbaugh, L.; Liu, K.; Bruss, V.; Sureau, C.; Hu, J. Common and Distinct Capsid and Surface Protein Requirements for Secretion of Complete and Genome-Free Hepatitis B Virions. J. Virol. 2018, 92 (14). https://doi.org/10.1128/JVI.00272-18.
(33) Luckenbaugh, L.; Kitrinos, K. M.; Delaney, W. E.; Hu, J. Genome-Free Hepatitis B Virion Levels in Patient Sera as a Potential Marker to Monitor Response to Antiviral Therapy. Journal of Viral Hepatitis. 2015, pp 561-570. https://doi.org/10.1111/jvh.12361.
(34) Sánchez-Báscones, E.; Parra, F.; Lobo-Castañón, M. J. Aptamers against Viruses: Selection Strategies and Bioanalytical Applications. Trends Analyt. Chem. 2021, 143, 116349.
(35) Kohlberger, M.; Gadermaier, G. SELEX: Critical Factors and Optimization Strategies for Successful Aptamer Selection. Biotechnol. Appl. Biochem. 2021. https://doi.org/10.1002/bab.2244.
(36) Valero, J.; Civit, L.; Dupont, D. M.; Selnihhin, D.; Reinert, L. S.; Idorn, M.; Israels, B. A.; Bednarz, A. M.; Bus, C.; Asbach, B.; Peterhoff, D.; Pedersen, F. S.; Birkedal, V.; Wagner, R.; Paludan, S. R.; Kjems, J. A Serum-Stable RNA Aptamer Specific for SARS-CoV-2 Neutralizes Viral Entry. Proc. Natl. Acad. Sci. U.S.A 2021, 118 (50), e2112942118.
(37) Maturana, C. J.; Verpeut, J. L.; Kooshkbaghi, M.; Engel, E. A. Novel Tool to Quantify with Single-Cell Resolution the Number of Incoming AAV Genomes Co-Expressed in the Mouse Nervous System. Gene Ther. 2021. https://doi.org/10.1038/s41434-021-00272-8.
(38) Brücher, D.; Kirchhammer, N.; Smith, S. N.; Schumacher, J.; Schumacher, N.; Kolibius, J.; Freitag, P. C.; Schmid, M.; Weiss, F.; Keller, C.; Grove, M.; Greber, U. F.; Zippelius, A.; Plückthun, A. IMATCH: An Integrated Modular Assembly System for Therapeutic Combination High-Capacity Adenovirus Gene Therapy. Mol Ther Methods Clin Dev 2021, 20, 572-586.
(39) Zhang, L.; Jackson, C. B.; Mou, H.; Ojha, A.; Peng, H.; Quinlan, B. D.; Rangarajan, E. S.; Pan, A.; Vanderheiden, A.; Suthar, M. S.; Li, W.; Izard, T.; Rader, C.; Farzan, M.; Choe, H. SARS-CoV-2 Spike-Protein D614G Mutation Increases Virion Spike Density and Infectivity. Nat. Commun. 2020, 11 (1), 6013.
(40) Song, Y.; Song, J.; Wei, X.; Huang, M.; Sun, M.; Zhu, L.; Lin, B.; Shen, H.; Zhu, Z.; Yang, C. Discovery of Aptamers Targeting Receptor-Binding Domain of the SARS-CoV-2 Spike Glycoprotein.
(41) Liu, Y. et al. Paired aptamer capture and FISH detection of individual virions enables cell-free determination of infectious titer. bioRxiv 2022.11.13.516306 (2022) doi: 10.1101/2022.11.13.516306.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of assaying a virion comprising:
a) capturing the virion on a solid substrate;
b) fixing the virion on the solid substrate;
c) washing the solid substrate; and
d) detecting the virion.

2. The method of claim 1, wherein virion is obtained from cells and wherein the solid substrate is a cell-free solid substrate.

3. The method of claim 1, wherein the solid substrate is a polystyrene plate or a cover glass.

4. The method of claim 1, wherein the step of capturing the virion on a solid substrate comprises:
a) coating the solid substrate;
b) immobilizing a binder to the coated solid substrate; and
c) contacting the solid substrate with the virion;
thereby capturing the virion on the solid substrate.

5. The method of claim 4, wherein the binder is a biotinylated binder, an aptamer, a nanoparticle, a lectin, a protein, or a chemical binder.

6. The method of claim 5, wherein the binder is an aptamer comprising from about 10 to about 60 nucleotides.

7. (canceled)

8. (canceled)

9. (canceled)

10. The method of claim 1, wherein the step of fixing the virion on the solid substrate comprises methanol fixation or crosslinking.

11. (canceled)

12. (canceled)

13. The method of claim 1, wherein the step of detecting the virion comprises use of an imaging modality, wherein the imaging modality comprises using probe sets in different fluorescent channels.

14. The method of claim 1, further comprising calculating virion titer.

15. The method of claim 14, wherein calculating virion titer comprises:
a) binarization;
b) colocalization; and
c) size thresholding.

16. (canceled)

17. The method of claim 1, wherein the virion is a DNA virus or an RNA virus.

18. (canceled)

19. The method of claim 1, wherein at least two types of virions are assayed.

20. The method of claim 19, wherein a ratio of the at least two types of virions is determined.

21. A kit for assaying a virion in a sample, the kit comprising:
a) a solid substrate;
b) a fixation solution; and
c) a wash solution.

22. The kit of claim 21, wherein the solid substrate is a polystyrene plate or a cover glass.

23. The kit of claim 21, wherein the solid substrate has an upper surface and a lower surface, wherein the upper surface is coated with streptavidin, thereby producing a streptavidin-coated upper surface of the solid substrate.

24. The kit of claim 23, wherein a biotinylated binder is bound to the streptavidin-coated upper surface of the solid substrate.

25. The kit of claim 22, wherein the biotinylated binder is an aptamer, a nanoparticle, a lectin, a protein, or a chemical binder.

26. (canceled)

27. (canceled)

28. (canceled)

29. (canceled)

30. The method of claim 1, wherein the step of detecting the virion comprises use of an imaging modality.

31. The method of claim 30, wherein the imaging modality is fluorescence in situ hybridization (FISH) selected from the group consisting of TurboFISH, smFISH, HCR-FISH, bDNA-FISH, or ClampFISH.
